(19) United States
(12) Patent Application Publication
(10) Pub. No.: US 2010/0115665 A1
(43) Pub. Date: May 6, 2010

(54) VIRUS TOLERANT PLANTS AND METHODS OF PRODUCING SAME

(76) Inventor: Hanokh Czosnek, Rehovot (IL)

Correspondence Address: FENNEMORE CRAIG, 3003 NORTH CENTRAL AVENUE, SUITE 2600, PHOENIX, AZ, 85012, US

(21) Appl. No.: 12/526,566

(22) PCT Filed: Feb. 3, 2008
(86) PCT No.: PCT/IL08/00146

§ 371 Date: Aug. 10, 2009

Related U.S. Application Data
(60) Provisional application No. 60/902,087, filed on Feb. 20, 2007.

(51) Int. Cl. A01H 1/00; A01H 5/00; A01H 5/10; C12N 5/04

(52) U.S. Cl. 800/279; 800/298; 800/301; 800/317.4; 800/317.3; 800/307; 800/317.2; 800/313; 800/312; 800/320; 800/323; 800/309; 800/314; 800/320.3; 800/320.1; 800/320.2; 800/306; 435/419

(57) ABSTRACT

The present invention relates to plants with enhanced tolerance to viral diseases, particularly to diseases caused by insect-transmitted viruses. The invention discloses transgenic plants expressing GroEL protein, and to engrafted plant comprising said transgenic plants or parts thereof, that are tolerant to insect-transmitted viral diseases. The present invention further discloses means and methods of producing same.

### FIELD OF THE INVENTION

The present invention relates to plants with enhanced tolerance to viral diseases, particularly to GroEl-expressing plants tolerant to diseases caused by viruses transmitted by insect vectors, and to means and methods of producing same.

### BACKGROUND OF THE INVENTION

Plant pathogenic viruses cause significant losses in agricultural fresh produce all over the world. Modern agricultural practices, including the growth of a single species in wide regions, and the demand for fresh produce all year round leading to an increase in greenhouse area, aggravated the problem of viral spread and increase the resultant damage. Various plant pathogenic viruses, including geminiviruses, which present one of the most serious disease problems in many vegetable crops in tropical and subtropical regions, are transmitted by insect vectors in a circulative manner.

Once ingested by their insect vector, the circulative viruses are not immediately available for infection. They need to be transferred from the digestive tract to the salivary glands of the insect vector, from which they are excreted with the saliva during the insect feeding on a plant (Brown J K and Czosnek H, 2000 In: Advances in Botanical Research. Plant Virus Vector Interactions: Plumb RT, Ed. Vol. 36. pp. 65-100. Academic Press). To avoid destruction in the haemolymph of their vector, these viruses interact with GroEL protein homologues produced by insect endosymbiotic bacteria.

GroELs are well-characterized proteins belonging to the chaperonin family. They are involved in many important biological processes such as protein post-translational folding and subunit assembly. In addition, chaperonins play a role in the infection cycle of insect-transmitted plant viruses (Mayer M P, 2005 Rev Physiol Biochem Pharmacol 153: 1-46). First shown for the Potato leafroll virus (PLRV), a circulative virus transmitted by the aphid Myzus persicae, the GroEL homologue produced by the insect primary endosymbiotic bacteria exhibited affinity for the virus. Interference in this interaction resulted in reduced capsid integrity and loss of infectivity (van den Heuvel J F J M et al., 1994 J Gen Virol 75: 2559-2565). The inventor of the present invention and co-workers have shown that Tomato yellow leaf curl virus (TYLCV), a geminivirus transmitted by the whitefly Bemisia tabaci, interacts in vivo with a GroEL produced by the insect endosymbiotic bacteria. Disturbing this interaction in vivo resulted in a dramatic reduction in TYLCV transmission (Morin S et al., 1999 Virology 256: 75-84). It was also shown that the capsid protein (CP) of TYLCV binds to B. tabaci GroEL in the yeast two-hybrid system (Morin S et al., 2000 Virology 276: 404-416). These phenomena were exploited to devise tools allowing trapping in vitro of plant viruses by either GroEL purified from the whitefly Bemisia tabaci or by whitefly GroEL over-expressed in E. coli (Akad F et al., 2004 Arch Virol 149: 1481-1497). Anti-GroEL antibody prevented TYLCV binding to GroEL. Viruses able to bind GroEL chaperonins are characterized as having a CP with a basic isoelectric point; a marked positive charge; being rich in arginine residues; and having a globular (or geminate) shape. For example in addition to several geminiviruses, GroEL was able to bind to a variety of RNA viruses such as Cucumber mosaic virus (CMV), Prune dwarf virus (PDV) and Tomato spotted wilt (TSWV), but not to Potato virus X and Potato virus Y (PVX and PVY), Grapevine leafroll virus (GLRV) and Tobacco mosaic virus (TMV).

The inventor of the present invention disclosed after the priority date of the present application that GroEL can also bind TYLCV in vivo: plant expressing GroEL exhibited mild or no symptoms upon whitefly-mediated inoculation of TYLCV (Akad F et al., 2007 Arch Virol 152: 1323-1339).

Controlling virus infection in plants is difficult, and is attempted by application of insecticides to destroy putative insect vectors and by breeding for resistance. In the last two decades, a genetic molecular approach has been taken to produce transgenic plants having increased tolerance or resistance to viral diseases that revealed a significant number of transgenic resistant plants.

For example, U.S. Pat. No. 7,294,758 discloses transgenic plants or plant tissue comprising polynucleotide encoding a non-mutated Rep protein of a tomato mottle geminivirus, wherein the plants show increased resistance to infection by a plant geminivirus. U.S. Pat. No. 6,291,743 discloses the production of transgenic plants containing DNA encoding AC1/C1 wild type and mutant sequences that negatively interfere in trans with geminiviral replication during infection, such that the resulting transgenic plants are resistant to geminivirus infection, and U.S. Pat. No. 6,747,188 discloses transgenic plants expressing a mutant AL3/C3 geminivirus protein, which increases resistance to infection by at least one geminivirus. U.S. Pat. No. 6,852,907 discloses methods for producing plant resistance to ssDNA virus, particularly a geminivirus such as mastrevirus, curtovirus or begomovirus, comprising introducing ssDNA-binding protein of the Inoviridae virus into the plant, wherein the ssDNA-binding protein is a phage coat protein, particularly, a coliphage gene 5 protein. However, most of the new plant varieties developed show resistance to a specific virus or to a small number of highly related virus species.

Viruses transmitted by insect vectors bind to GroEL homologues expressed by endosymbiotic bacteria present within the insect host. This phenomenon has been suggested as a general mechanism shared by plant circulative viruses to avoid destruction in the insect's haemolymph (Gibbs M, 1999 Nature 399: 415).

However, nowhere in the background art is it taught or suggested that virus resistance may be conferred to plants by expressing GroEL in the

Thus, there is a great need for, and it would be highly advantageous to have, means and methods for developing plants with increased tolerance to a plurality of virus types. The use of such plants will decrease the need to spray insecticides, which need to be highly restricted due to the environmental hazards involved.

### SUMMARY OF THE INVENTION

The present invention relates to plants resistant to diseases caused by viruses transmitted by insect vectors. Particularly, the present invention provides transgenic plants expressing GroEL homologous protein in a tissue specific manner, the GroEL protein serving as a virus trap within the plant, particularly within the plant phloem.

Unexpectedly, the present invention now shows that plants transformed with a polynucleotide encoding a GroEL (i) express active GroEL protein capable of interacting with circulative viruses and (ii) show enhanced tolerance to diseases caused by the circulative viruses. Furthermore, the present invention shows that GroEL protein can be transmitted from a transgenic rootstock to a non-transgenic graft.

Without wishing to be bound by any specific mechanism of action or theory, the enhanced resistance to the viral disease may be attributed to the capture of the virus by the GroEL protein, which prevents the virus spread in the plant thereby limiting the disease symptoms.

Thus, according to one aspect, the present invention provides a transgenic plant comprising at least one cell transformed with a polynucleotide encoding a GroEL protein, an active fragment, variant or homologue thereof.

According to certain embodiments, the polynucleotide encodes any one of GroEL proteins capable of binding to the capsid protein (CP) of a plant pathogenic virus. According to one embodiment, the CP protein is characterized by having a basic isoelectric point, a marked positive charge, and a high percentage of basic (e.g. arginine) amino acid residues. According to typical embodiments, the polynucleotide encodes any one of GroEL proteins that can be found in endosymbiotic bacteria of insects that transmit viruses in a circulative manner.

According to one embodiment, the polynucleotide encodes Bemisia tabaci GroEL protein. According to certain typical embodiments, the GroEL protein is encoded by a polynucleotide comprising a nucleic acid sequence at least 70%, at least 75%, at least 80%, at least 85%, at least 90%, or at least 95% homologous to SEQ ID NO:1. According to one embodiment, the polynucleotide comprises a nucleic acid sequence as set forth in SEQ ID NO:1. According to other typical embodiments, the encoded protein has an amino acid sequence having at least 70%, at least 75%, at least 80%, at least 85%, at least 90%, at least 95% homology to SEQ ID NO:2, or having the amino acids sequence as set forth in SEQ ID NO:2 (GenBank accession numbers AF130421, AY462929-32 or AY445872-4).

It is to be understood that according to the teachings of the present invention, the functional part of the GroEL protein is the domain(s) involved in the binding of a plant virus. Accordingly, any polynucleotide encoding GroEL having a virus-binding ability can be employed, including plant endogenous polynucleotide encoding proteins homologous to GroEL proteins. In addition, the polynucleotide can encode a fragment, variant or homologue of GroEL as long as the virus-binding activity of the protein is preserved.

According to certain embodiments, the polynucleotide encodes an active fragment of GroEL, wherein the fragment maintains the virus binding properties of the parent protein. According to certain embodiments, the polynucleotide encodes a peptide having a length of at least 10 amino acids.

According to certain embodiments, the transgenic plant has an enhanced tolerance to at least one disease caused by an insect-transmitted plant pathogenic virus compared to a non-transgenic plant. According to typical embodiments, the virus may bind to a member of the GroEL protein family. According to some embodiments, the virus is selected from the group present in Table 1. It is to be understood that the list in Table 1 is given merely as a set of examples and additional viruses that may be bound by the GroEL protein as disclosed herein are explicitly encompassed in the present invention.

According to certain embodiments, the virus is selected from the group consisting of geminiviruses: Tomato Yellow Leaf Curl Virus (TYLCV), RNA viruses: Cucumber mosaic virus (CMV), Prune dwarf virus (PDV) and Tomato spotted wilt (TSWV).

According to specific embodiments, the virus is selected from the group consisting of TYLCV and CMV.

Any plant susceptible to an insect-transmitted plant virus as described herein above may be used according to the teachings of the present invention. This is a wide group of plants, including, but not limited to, tomato, tobacco, cucumber, prune, potato, bean, barley, soybean, pea, beet, grapevine, petunia, abutilon, melon, watermelon, okra, cotton, cassava, wheat, maize, rice and cabbage.

According to yet other embodiments, the polynucleotides of the present invention are incorporated in a DNA construct enabling their expression in the plant cell. According to one embodiment, the DNA construct comprises at least one expression regulating element selected from the group consisting of a promoter, an enhancer, an origin of replication, a transcription termination sequence, a polyadenylation signal and the like.

According to some embodiments, the DNA construct comprises a promoter. The promoter can be constitutive, induced or tissue specific promoter as is known in the art. According to certain embodiments, the promoter is a tissue specific promoter operable in a plant cell. According to typical embodiment, the promoter is phloem-specific promoter. According to other embodiments, the DNA construct further comprises transcription termination and polyadenylation sequence signals.

Optionally, the DNA construct further comprises a selectable marker, enabling a convenient selection of the transformed cell/plant. Additionally or alternatively, a reporter gene can be incorporated into the construct, as to enable selection of transformed cells or plants expressing the reporter gene. According to one embodiment, the selection marker is a gene inducing antibiotic resistance within the plant. According to one embodiment, the antibiotic is selected from the group consisting of carbenicillin and kanamaycin.

The polynucleotides of the present invention and/or the DNA constructs comprising same can be incorporated into a plant transformation vector.

The present invention also encompasses seeds of the transgenic plant, wherein plants grown from said seeds comprise at least one cell having enhanced tolerance to at least one disease caused by an insect-transmitted plant pathogenic virus compared to a non-transgenic plant. The present invention further encompasses fruit, leaves or any part of the transgenic plant, as well as tissue cultures derived thereof and plants regenerated therefrom.

The present invention also relates to engrafted plants comprising a transgenic viral-resistant rootstock and a susceptible graft, wherein the entire plant is resistant to viral disease.

According to this aspect of the invention, there is provided an engrafted plant comprising a transgenic rootstock comprising at least one cell transformed with a polynucleotide encoding GroEL protein, or an active fragment, variant or homologue thereof, and a non-transgenic graft, wherein the entire engrafted plant has an enhanced tolerance to at least one disease caused by an insect-transmitted plant pathogenic virus.

Without wishing to be bound by any specific mechanism or theory, the enhanced resistance of the whole engrafted plant may be the result of the translocation of GroEL protein homologue expressed by the transgenic rootstock to the non-transgenic graft, optionally via the plant vascular system, particularly the phloem. It should be noted that the GroEL protein can be found in the non-germinal-originating tissues of the grafted plant, i.e. the protein could not be detected in seeds, pollen or fruit. Moreover, the protein seems to be found in the vascular system only, and it does not enter the plant gametes.

Such engrafted plants have high commercial value since agricultural products, particularly fruit obtained from transgenic plants are not desired in many countries. In the engrafted plants of the present invention, the whole plant is tolerant to viral diseases and thus can produce a normal high yield, while the fruit are obtained from the non-transgenic graft and can be marketed as non-genetically modified (GMO) products.

The present invention also relates to a method of producing a transgenic plant having an enhanced tolerance to at least one disease caused by an insect-transmitted plant pathogenic virus compared to a corresponding non-transgenic plant. The method comprises (a) transforming a plant cell with a polynucleotide encoding GroEL protein or an active fragment, variant or homologue thereof; and (b) regenerating the transformed cell into a plant having an increased tolerance to at least one disease caused by an insect-transmitted plant pathogenic virus as compared to a corresponding non-transgenic plant.

The exogenous polynucleotide(s) encoding GroEL homologue according to the teachings of the present invention can be introduced into a DNA construct to include the entire elements necessary for transcription and translation as described above, such that the polypeptides are expressed within the plant cell.

Transformation of plants with a DNA construct may be performed by various means, as is known to one skilled in the art. Common methods are exemplified by, but are not restricted to, Agrobacterium-mediated transformation, microprojectile bombardment, pollen mediated transfer, plant RNA virus mediated transformation, liposome mediated transformation, direct gene transfer (e.g. by microinjection) and electroporation of compact embryogenic calli. According to one embodiment, the transgenic plants of the present invention are produced using Agrobacterium mediated transformation.

Transgenic plants comprising the construct of the present invention may be selected employing standard methods of molecular genetics, as are known to a person of ordinary skill in the art. According to one embodiment, the transgenic plants are selected according to their resistance to an antibiotic. According to certain embodiments, the antibiotic serving as a selectable marker is one of the group consisting of carbenicillin and kanamycin. Additionally or alternatively, the transgenic plant of the present invention can be selected according to their resistance to at least one GroEL-binding virus. Typically, resistance is examined by deliberate inoculation of the plant with the virus and monitoring the development of disease systems by means specific to the disease as is known to a person skilled in the art.

According to another aspect the present invention relates to the transgenic plants generated by the methods of the present invention as well as to their seeds, fruits, roots and other organs or isolated parts thereof.

It is to be understood explicitly that the scope of the present invention encompasses homologues, analogs, variants and derivatives, including shorter and longer polypeptides, proteins and polynucleotides, as well as polypeptide, protein and polynucleotide analogs with one or more amino acid or nucleic acid substitution, as well as amino acid or nucleic acid derivatives, non-natural amino or nucleic acids and synthetic amino or nucleic acids as are known in the art, with the stipulation that these variants and modifications must preserve the virus binding activity of the GroEL polypeptide in the context of the present invention of enhancing the tolerance of the transformed cell to an insect-transmitted viral disease. Specifically, any active fragments of the polypeptide or protein as well as extensions, conjugates and mixtures are disclosed according to the principles of the present invention.

Other objects, features and advantages of the present invention will become clear from the following description and drawings.

### BRIEF DESCRIPTION OF THE FIGURES

FIG. 1 shows a scheme of Agrobacterium tumefaciens binary vector pEP/GroEL used to transform tomato plants with the GroEL gene from the whitefly Bemisia tabaci.

FIG. 2 shows the presence of GroEL DNA (FIG. 2A) and GroEL protein (FIGS. 2B,C) in tomato plants regenerated following co-cultivation of explants with Agrobacterium containing the binary vector pEP/GroEL (“T₀” generation). FIG. 2A: DNA of selected plants was subjected to PCR using GroEL-specific primers; transgenic plants yielded the expected ˜500 by product. (a): agarose gel showing PCR products obtained with a selected number of plants; 0: MP1 plant from seed, P: plasmid containing the GroEL clone, M: 100 by ladder, arrow head: 500 bp. (b): the gel was blotted on a membrane and subjected to hybridization with a GroEL-specific probe. FIG. 2B: Western blot analysis of selected regenerated plants using a GroEL specific antibody; GroEL: protein over-expressed in E. coli. FIG. 2C: immunolocalization of GroEL in tissue prints; arrow head points to label in the inner phloem. Note that the non-transgenic regenerated plant 44 is not expressing GroEL. *: regenerated plants of the MP1 genotype.

FIG. 3 represents resistance of “T₀” plants to whitefly-mediated TYLCV infection. FIG. 3A: PCR amplification of TYLCV DNA from selected regenerated transgenic and non-transgenic tomato plants, two weeks after whitefly-mediated inoculation; 10 ng DNA from a young leaf of infected and non-infected plants were used as template and the products were analyzed after 30 cycles; F1 and MP1: inoculated plants grown from seeds, 0: non-infected MP1 plant, P: plasmid pTYJ20.4 containing the cloned full-length TYLCV genome; M: 100 by molecular weight ladder; 500 by is indicated by a star. FIG. 3B: Following inoculation, the severity of symptoms (disease severity index, DSI) was scored 28, 40 and 60 days post inoculation (dpi); note that regenerated plants 27 and 44 were not transgenic. *: plants of the MP1 genotype. FIG. 3C: absence of disease symptoms at 90 dpi in infected transgenic plants from MP1 and F1 genotype; compared with strong disease symptoms presented by infected non-transgenic plants.

FIG. 4 shows whitefly GroEL DNA and protein in transgenic plants from the “T₁” generation and viral DNA amounts upon inoculation. FIG. 4A: Southern blot hybridization of EcoRI-digested plant DNA with radiolabeled GroEL gene; arrow head: 4,200 by DNA fragment containing the GroEL gene flanked by SUC2 promoter and the NOS terminator. FIG. 4B: presence of the GroEL protein as assayed by Western blot in selected plants using the GroEL specific antibody; note that plant 44 is not transgenic. *: plants of the MP1 genotype. FIG. 4C: Comparison of viral DNA amounts at 60 dpi in infected transgenic T₁ plants 15A and 30D and in infected non-transgenic MP1 and F1 plants by semi-quantitative PCR analysis of viral DNA (full arrow); β-actin served as internal standard (empty arrow); 50 ng plant DNA was used as template; the reactions were sampled after the number of cycles indicated and the products subjected to agarose gel electrophoresis. M: 100 by ladder, star: 500 bp.

FIG. 5 shows the detection of virus bound to GroEL in the sap of “T₁” transgenic plants. Sap of infected transgenic plants 15A, 43C and 30B (at 60 dpi) was added to PCR tubes coated with anti-GroEL antibody (case A) and to uncoated tubes (case B); similarly extracts of non-inoculated transgenic plants 15D and 43G (case C), extracts of infected non-transgenic plants 11 and 12 (case D), and mixtures of non-infected transgenic plants 15D and 43G with infected non-transgenic plants 11 and 12 (case E), were incubated in PCR tubes coated with anti-GroEL antibody. After thorough washes, a PCR mix containing TYLCV-specific primers was added to the tube and the PCR products were analyzed by agarose gel electrophoresis. M: 100 by ladder, arrow head: 500 bp.

FIG. 6 demonstrates acquisition of TYLCV from resistant “T₂” plants by whiteflies and transmission to tomato test plants. FIG. 6A: PCR detection of viral DNA in infected “T₂” plants 3, 7 and 62 and in non-infected “T₂” plant 68; 144 is infected non-transgenic FA144 cultivar. FIG. 6B: PCR detection of viral DNA in whiteflies that fed on plants in A. FIG. 6C: PCR detection of viral DNA in test plants inoculated by whiteflies fed on resistant “T₂” plants in A. (3 target test plants per source plant, except for 144). M: 100 by ladder, star: 500 bp.

FIG. 7 shows behavior of non-transgenic susceptible tomato plants FA144 grafted on resistant GroEL-transgenic “T₂” rootstocks (scions). Specification of graft and scion is graft/scion. FIG. 7A: Presence of GroEL in tomato plants serving as scions, detecting by Western blotting using a GroEL antibody. FIG. 7B: Disease severity index of grafts, 20, 40 and 60 days after whitefly-mediated inoculation; infected non-transgenic MP (MP1) and FA144 (144) plants served as susceptible controls; infected 15A and 30D “T₁″resistant plants served as resistant controls. FIG. 7C: Presence of severe disease symptoms presented by FA144 plant grafted on GroEL-producing resistant ‘T₂” plant 33 (144/33) at 60 dpi; note the very mild symptoms presented by resistant ‘T₂″ plant 16 grafted on susceptible non-transgenic FA144 scion (16/144).

FIG. 8 shows Translocation of GroEL from transgenic scions to non-transgenic grafts and detection of GroEL-TYLCV complexes in the grafts. FIG. 8A Left panel: immunodetection of GroEL in FA144 plants grafted on scions of GroEL-expressing “T₂”′ plants 16 (144/16) and 43 (144/43); right panel: appearance of severe symptoms in FA144 plants grafted on plant 16, and of very mild symptoms on plant 16 grafted on 144. FIG. 8B: Detection of GroEL-TYLCV complexes in inoculated FA144 grafts (see also FIG. 5); sap from FA144 grafts (grafted on plants 16, 43, 28 and 33: 144/16, 144/43, 144/28, 144/33) was incubated in PCR tubes coated or not coated with anti-GroEL antibody; after washing, a PCR mix containing TYLCV-specific primers was added and the PCR products were analyzed; 0 no template, 1, 2 coated tubes without sap, 16 and 43 infected non-grafted plants, M 100-bp molecular marker, star: 500 bp.

FIG. 9 shows the presence of GroEL and NptII genes and detection of GroEL expression in transgenic N. benthamiana plants. FIG. 9A: Agarose gel electrophoresis showing PCR products of amplification of GroEL and NptII genes from five different transgenic plants numbered 1-5, and from two non-transgenic wild-type plants (WT) numbered 6-7. FIG. 9B: Western blot analysis of proteins extracted from leaves of three transgenic tobacco and two non-transgenic tobacco plants, respectively numbered 1-3 and 4-5, and immunodetected with anti-GroEL antibodies. M: marker, 100 by DNA ladder. P: Cloned genes.

FIG. 10 shows tolerance of GroEL-expressing N. benthamiana to TYLCV. FIG. 10A: Assessment of infection of non-transgenic wild type (WT 1, 2) and transgenic (T 1, 2) plants at 7 dpi, by PCR (left panel) using specific primers and by Western blot analysis (right panel) using antibodies against the virus CP (*: infected plant). 0: no template; M: marker, 100 by DNA ladder. FIG. 10B: Detection of TYLCV-GroEL binding in the sap of transgenic plants at 7 dpi; detection of viral DNA by PCR (left panel) and of viral CP by Western blot analysis (right panel). +: infected tobacco plant. FIG. 10C: TYLCV symptoms in infected transgenic and non-transgenic plants at 25 dpi.

FIG. 11 shows tolerance of GroEL-expressing N. benthamiana plants to CMV. FIG. 11A: Assessment of infection of non-transgenic wild type (WT 1, 2) and transgenic (T 1, 2) plants at 5 dpi, by RT-PCR (left panel) using specific primers and by Western blot (right panel) using antibodies against the virus coat protein CP (*: infected plant). 0: no template; M: marker, 100 by DNA ladder. FIG. 11B: Detection of CMV-GroEL binding in the sap of transgenic plants at 5 dpi; detection of viral RNA by RT-PCR (left panel) and of viral CP by Western blot analysis (right panel). +: RNA from inoculated plant; FIG. 11C: Appearance of CMV symptoms in infected transgenic and non-transgenic plants at 100 dpi.

FIG. 12 shows that GroEL-expressing N. benthamiana plants are susceptible to GVA. FIG. 12A: Assessment of infection of wild type (WT 1, 2) and transgenic (T 1, 2) plants by RT-PCR (left panel) at 5 dpi using specific primers and by Western blot (right panel) using antibodies against the virus coat protein (CP) (*: infected plant). FIG. 12B: Detection of CMV-GroEL binding in the sap of transgenic plants at 7 dpi; detection of viral RNA by RT-PCR (left panel) and of viral CP by Western blot analysis (right panel). +: RNA from inoculated plant; 0: no template; M: marker, 100 by DNA ladder. FIG. 12C: GVA symptoms in infected transgenic and non-transgenic plants at 20 dpi.

FIG. 13 shows that GroEL-expressing N. benthamiana plants are susceptible to TMV. FIG. 13A: Assessment of infection of wild type and transgenic plants by RT-PCR (left panel) at 5 dpi using specific primers and by Western blot analysis (right panel) using antibodies against the virus CP; *: infected plant. FIG. 13B: Detection of TMV-GroEL binding in the sap of wild type (WT 1, 2) and transgenic (T 1, 2) plants at 7 dpi; detection of viral RNA by RT-PCR (left panel) and of viral CP by Western blot analysis (right panel). +: RNA from inoculated plant; 0: no template; M: marker, 100 by DNA ladder. FIG. 13C: TMV symptoms in infected transgenic and non-transgenic plants at 6 dpi.

### DETAILED DESCRIPTION OF THE INVENTION

The present invention answers the need for plants, particularly agricultural plants, which are resistance or show enhanced tolerance to a variety of viral diseases. Particularly, the present invention provides plants having enhanced tolerance to diseases caused by viruses transmitted by insect vectors.

### DEFINITIONS

The term “plant” is used herein in its broadest sense. It includes, but is not limited to, any species of woody, herbaceous, perennial or annual plant. It also refers to a plurality of plant cells that are largely differentiated into a structure that is present at any stage of a plant's development. Such structures include, but are not limited to, a root, stem, shoot, leaf, flower, petal, fruit, etc. The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant organ or a plant tissue.

As used herein, the term “engrafted plant” refers to a plant comprising a rootstock (also referred to as scion) and a graft, wherein the graft is engrafted onto the rootstock by any method known in the art.

As used herein, the terms “rootstock” or “scion” refers to a stock for grafting comprising the root part of a plant. The term “graft” refers to a detached living portion of a plant designed or prepared for union with a stock in grafting, usually supplying solely or predominantly aerial parts to the engrafted plant.

As used herein, the term “virus” refers to a plant virus, i.e. a virus capable of infecting a plant cell and propagating within the plant cell. Typically, the virus is pathogenic, such that substantial viral infection causes disease symptoms and yield lose in agricultural crops.

As used herein, the term “GroEL protein” refers to a family of chaperonin proteins found in insect's endosymbiotic bacteria, but also in free bacteria such as Escherichia coli and Agrobacterium tumefaciens, as well as human pathogenic bacteria such as Mycoplasma genitalium, Neisseria gonorrhoeae and Salmonella. typhi. The GroEL protein is required for the proper folding of many proteins in the bacteria. To function properly, GroEL requires the lid-like co-chaperonin protein complex GroES. In eukaryotes, the proteins Hsp60 and Hsp10 are structurally and functionally nearly identical to GroEL and GroES, respectively. Properties and list of proteins belonging to this family can be found in the URL: http://en.wikipedia.org/wiki/GroEL and the relevant links, as defined therein (see also Farres M A et al., 2005 Journal of Evolutionary Biology 18: 651-660). More specifically the term refers to GroELs that can be found in endosymbiotic bacteria of insects that transmit viruses in a circulative manner, capable of binding the transmitted viruses. The sequence of the GroEL from the whitefly Bemisia tabaci can be found in GenBank accession number AF130421 and as shown in Morin S et al., (1999 and 2000, ibid) and in GenBank accession numbers AY462929 and AY445872 (Tan Z et al., 2004 Secondary endosymbiont of Bemisia tabaci GroEL (GroEL) gene, NCBI).

The terms “GroEL homologue” refers to proteins and polynucleotides encoding same that are at least 70%, at least 75%, at least 80%, at least 85%, at least 95% or more homologous to GroEL proteins found in insect endosymbiotic bacteria.

The term “homology”, as used herein, refers to a degree of sequence similarity in terms of shared amino acid or nucleotide sequences. There may be partial homology or complete homology (i.e., identity). For amino acid sequence homology, amino acid similarity matrices may be used as are known in different bioinformatics programs (e.g. BLAST, Smith Waterman). Different results may be obtained when performing a particular search with a different matrix. Homologous peptide or polypeptides are characterized by one or more amino acid substitutions, insertions or deletions, such as, but not limited to, conservative substitutions, provided that these changes do not affect the biological activity of the peptide or polypeptide as described herein.

Degrees of homology for nucleotide sequences are based upon identity matches with penalties made for gaps or insertions required to optimize the alignment, as is well known in the art (e.g. Altschul S F et al., 1990, J Mol Biol 215(3): 403-10; Altschul S F et al., 1997, Nucleic Acids Res. 25: 3389-3402). The degree of sequence homology is presented in terms of percentage, e.g. “70% homology”. As used herein, the term “at least” with regard to a certain degree of homology encompasses any degree of homology from the specified percentage up to 100%.

The term “GroEL fragment” as used herein refers to sub-sequences (fragments) of the GroEL protein that still maintains the virus binding properties of the parent protein. The fragment may correspond to a contiguous amino acid sequence of the complete protein, or it may be formed from non-contiguous amino acids sequences. For example, mutational analysis experiments of the gene encoding Buchnera GroEL of Myzus persicae (MpB GroEL) revealed that the determinants required for Potato Leaf Virus (PLRV) binding are located in the equatorial domain. The equatorial domain forms the waist of the GroEL 14-mer and holds the protein cylinder together. It is made up of two regions at the N-terminus and C-terminus that are not contiguous in the amino acid sequence but are in spatial proximity after folding of the GroEL polypeptide (Hogenhout S A et al., 2000 J. of Virol 74: 4541-4548). Typically, these are peptides having a length of from at least 10 amino acids. According to certain embodiments, the protein fragments are in a length of from about 10 amino acids to about 50 amino acids. Any person skilled in the art can easily prepare a series of protein fragments and determine by simple binding assay which of the fragments binds to the virus CP and the affinity of binding and thus identify the fragments that fall under the scope of the present invention.

The term “GroEL variant” as used herein refers to a virus-binding variant of the protein and relates to the fact that not all amino acids of the complicated GroEL chaperonin protein are involved in the virus binding property (most are in fact involved in the chaperonin activity). Therefore, amino acid(s) (whether continuous in the secondary structure of the protein, or whether brought together spatially by the protein folding) which are not involved in the virus binding can be easily replaced, or modified, or eliminated as long as the virus binding regions are maintained either unaltered, or the amino acids in the virus binding regions are replaced with conservative substitutions. Any sequence coding for modified (variant) GroEL protein that still maintains the virus binding properties of the parent protein can be used to transform plants of the present invention. As describe hereinabove, only the equatorial domain of the GroEL homolog (composed of the non-continuous N- and C-terminal present in a vicinity in the folded protein) are required for viral binding, distinct from the apical domain involved in protein binding.

As used herein, the term “GroEL-binding virus” refers to any plant-infecting virus, that is transmitted in a circulative manner by its insect host, and that may bind in vivo and in vitro to a member of the GroEL protein family. A non-exhaustive list of such viruses is given in Table 1 hereinbelow. Typically these viruses have a capsid protein (CP) with a basic isoelectric point, a marked positive charge, are rich in arginine residues and the particle has a globular (or geminate) shape.

TABLE 1
GroEL-binding viruses

| | Virus name | | Properties of | | | | |
|---|---|---|---|---|---|---|---|
| | Acronym, | | Coat Protein | | | | Binding |
| | Accession | Virion | | | | Arg | to |
| Genus | No. | Shape | M.W. | Pi | Charge | (%) | GroEL |
| Begomovirus | Tomato | Geminate | 30,285 | 10.4 | 22.7 | 12.9 | Yes^{a} |
| | yellow leaf | | | | | | |
| | curl virus | | | | | | |
| | TYLCV, | | | | | | |
| | X1565 | | | | | | |
| Begomovirus | Abutilon | Geminate | 28,052 | 10.0 | 17.8 | 11.3 | Yes^{a} |
| | mosaic | | | | | | |
| | virus | | | | | | |
| | AbMV, | | | | | | |
| | NC_001928 | | | | | | |
| Begomovirus | African | Geminate | 30,129 | 10.3 | 22.8 | 12.5 | Yes^{a} |
| | cassava | | | | | | |
| | mosaic virus | | | | | | |
| | ACMV, | | | | | | |
| | AF366902 | | | | | | |
| Cucumovirus | Cucumber | Globular | 24,113 | 10.3 | 12.2 | 13.0 | Yes^{a} |
| | mosaic | | | | | | |
| | virus | | | | | | |
| | CMV, | | | | | | |
| | D10538 | | | | | | |
| Luteovirus | Bean leafroll | Globular | 21,966 | 11.2 | 22.3 | 13 | Yes^{b} |
| | virus | | | | | | |
| | BLRV, | | | | | | |
| | NC_003369 | | | | | | |
| Luteovirus | Barley | Globular | 21,930 | 12.1 | 23.2 | 15.0 | Yes^{b} |
| | yellow dwarf | | | | | | |
| | virus | | | | | | |
| | BYDV, | | | | | | |
| | NC_002160 | | | | | | |
| Luteovirus | Cucurbit | Globular | 22,160 | 12.1 | 23.2 | 16.9 | Yes^{b} |
| | aphid-borne | | | | | | |
| | yellows virus | | | | | | |
| | CABYV, | | | | | | |
| | NC_00368 | | | | | | |
| Luteovirus | Soybean | Globular | 22,201 | 11.3 | 22.3 | 13.5 | Yes^{b} |
| | dwarf | | | | | | |
| | virus | | | | | | |
| | SbDV, | | | | | | |
| | NC_003056 | | | | | | |
| Luteovirus | Pea enation | Globular | 21,104 | 11.2 | 19.3 | 15.6 | Yes^{b} |
| (Enamovirus) | mosaic | | | | | | |
| | virus | | | | | | |
| | PEMV, | | | | | | |
| | NC_003629 | | | | | | |
| Luteovirus | Potato | Globular | 23,127 | 11.6 | 24.2 | 15.5 | Yes^{b} |
| (Polerovirus) | leafroll | | | | | | |
| | virus | | | | | | |
| | PLRV, | | | | | | |
| | NC_001747 | | | | | | |
| Luteovirus | Beet western | Globular | 22,459 | 11.7 | 22.2 | 16.0 | Yes^{b} |
| (Polerovirus) | yellows virus | | | | | | |
| | BWYV, | | | | | | |
| | NC_003743 | | | | | | |
| Potexvirus | Potato | Filamentous | 25,111 | 7.0 | 0.06 | 5.3 | No^{a} |
| | virus X | | | | | | |
| | PVX, | | | | | | |
| | AF260641 | | | | | | |
| Potyvirus | Potato | Filamentous | 29,879 | 5.9 | −3.5 | 7.0 | No^{a} |
| | virus Y | | | | | | |
| | PVY, | | | | | | |
| | M95491 | | | | | | |
| Trichovirus | Grapevine | Filamentous | 21,624 | 8.4 | 1.2 | 6.9 | No^{a} |
| | virus A | | | | | | |
| | GVA, | | | | | | |
| | NC_003604 | | | | | | |
| Tospovirus: | Tomato | Isometric | 28897.5 | 9.78 | 7.45 | 3.64 | Yes^{a} |
| Bunyaviridae | spotted | | | | | | |
| | wilt virus | | | | | | |
| | TSWV, | | | | | | |
| | D13926 | | | | | | |
| Closterovirus | Grapevine | Isometric | 34802.8 | 6.78 | −0.4 | 3.0 | No^{a} |
| | leafroll virus | | | | | | |
| | GLRV, | | | | | | |
| | 3NC_004667 | | | | | | |
| Llavirus: | Prune dwarf | Isometric | 23922.2 | 10.0 | 9.0 | 6.26 | Yes^{a} |
| Bromoviridae | virus | | | | | | |
| | PDV, | | | | | | |
| | U31310 | | | | | | |
| Nepovirus: | Tobacco | Isometric | 57177.8 | 7.25 | 1.78 | 6.03 | No^{a} |
| Comoviridae | ringspot | | | | | | |
| | virus | | | | | | |
| | TRSV, | | | | | | |
| | AF461164 | | | | | | |
| Tobamovirus | Tobacco | Filamentous | 17620.0 | 4.83 | −2.04 | 9.36 | No^{a} |
| | mosaic virus | | | | | | |
| | TMV, | | | | | | |
| | NC_001367 | | | | | | |

^{a}From Akad et al., 2004
^{b}From the literature
^{c}Retrieved from GenBank and analyzed using the DNAMAN software

The terms “plant with enhanced tolerance to viral disease” and “tolerant plant” or “resistant plant” are used interchangeably to refer to a plant having an increased tolerance to the virus compared to a non-resistant (susceptible) plant. The increased tolerance may be examined by deliberate infection of the plant with the virus in question. Plants showing lower symptom intensity compared to susceptible plant, according to a symptom scale specific for each virus, are defined as plants resistant to the virus. Optionally or alternatively, the titer of the virus found in the infected plant can be measured, wherein tolerant plant show lower titer, particularly during the first 2 weeks of infection, compared to susceptible plants

These terms do not necessarily mean total immunity from all damages of viral infection but merely a decrease as compared to non-manipulated control. Most importantly, the terms refer to plants having similar yields compared to non-infected plants. To the contrary, susceptible plants are defined by an almost total yield loss as a result of viral infection.

The tolerant plants as defined above can be transgenic as well as engrafted non-transgenic plants. Resistance can be a stable trait, which can be inherited to the offspring population. Alternatively, resistance exists only as long as the engrafted plant comprises a rootstock and a graft. In the later situation, a plant resistant to a viral disease is also referred to as a plant protected from the viral disease.

The non-transgenic engrafted graft, to which the resistance is conferred, does not have to be of the same variety as the transgenic plant onto which it is grafted. This means that a single transgenic plant variety (for example a specific tomato variety) can be used to confer viral resistance to many varieties of non-transgenic plants (other varieties of tomato) grafted on it.

The term “gene” refers to a nucleic acid (e.g., DNA or RNA) sequence that comprises coding sequences necessary for the production of RNA or a polypeptide. A polypeptide can be encoded by a full-length coding sequence or by any part thereof. The term “parts thereof” when used in reference to a gene refers to fragments of that gene, particularly fragment encoding a GroEL protein fragment as is defined hereinabove. The fragments may range in size from a few nucleotides to the entire gene sequence minus one nucleotide. Thus, “a nucleic acid sequence comprising at least a part of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated (or untranslated) sequences (5′ UTR). The sequences which are located 3′ or downstream of the coding region and which are present on the mRNA are referred to as 3′ non-translated (or untranslated) sequences (3′ UTR).

The term “nucleic acid” as used herein refers to RNA or DNA that is linear or branched, single or double stranded, or a hybrid thereof. The term also encompasses RNA/DNA hybrids.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA.

The term “construct” as used herein refers to an artificially assembled or isolated nucleic acid molecule which includes the gene of interest. In general a construct may include the gene or genes of interest, a marker gene which in some cases can also be the gene of interest and appropriate regulatory sequences. It should be appreciated that the inclusion of regulatory sequences in a construct is optional, for example, such sequences may not be required in situations where the regulatory sequences of a host cell are to be used. The term construct includes vectors but should not be seen as being limited thereto.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

The terms “promoter element,” “promoter,” or “promoter sequence” as used herein, refer to a DNA sequence that is located at the 5′ end (i.e. precedes) the protein coding region of a DNA polymer. The location of most promoters known in nature precedes the transcribed region. The promoter functions as a switch, activating the expression of a gene. If the gene is activated, it is said to be transcribed, or participating in transcription. Transcription involves the synthesis of mRNA from the gene. The promoter, therefore, serves as a transcriptional regulatory element and also provides a site for initiation of transcription of the gene into mRNA. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters which cause a gene to be expressed in most cell types at most times are commonly referred to as “constitutive promoters”. Promoters that derive gene expression in a specific tissue are called “tissue specific promoters. Tissue specific promoters can be expressed constitutively or their expression may require a specific induction. New promoters of various types useful in plant cells are constantly being discovered; numerous examples may be found in the compilation by Okamuro J K and Goldberg R B 1989 In: Marcus A Ed. The Biochemistry of Plants: A comprehensive Treatise. Vol. 15 Molecular Biology Academic Press 1-82 (see also Shahmuradov, I A et al. 2003 Nucleic Acids Research 31: 114-117).

As used herein, the term an “enhancer” refers to a DNA sequence which can stimulate promoter activity, and may be an innate element of the promoter or a heterologous element inserted to enhance the level or tissue-specificity of a promoter.

The term “expression”, as used herein, refers to the production of a functional end-product e.g., an mRNA or a protein.

The terms “heterologous gene” or “exogenous genes” refer to a gene encoding a factor that is not in its natural environment (i.e., has been altered by the hand of man). For example, a heterologous gene includes a gene from one species introduced into another species. A heterologous gene also includes a gene native to an organism that has been altered in some way (e.g., mutated, added in multiple copies, linked to a non-native promoter or enhancer sequence, etc.). Heterologous genes may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). Heterologous plant genes are distinguished from endogenous plant genes in that the heterologous gene sequences are typically joined to nucleotide sequences comprising regulatory elements such as promoters that are not found naturally associated with the gene for the protein encoded by the heterologous gene or with plant gene sequences in the chromosome, or are associated with portions of the chromosome not found in nature (e.g., genes expressed in loci where the gene is not normally expressed). A plant gene endogenous to a particular plant species (endogenous plant gene) is a gene which is naturally found in that plant species or which can be introduced in that plant species by conventional breeding.

The term “transgenic” when used in reference to a plant or seed (i.e., a “transgenic plant” or a “transgenic seed”) refers to a plant or seed that contains at least one heterologous gene in one or more of its cells. The term “transgenic plant material” refers broadly to a plant, a plant structure, a plant tissue, a plant seed or a plant cell that contains at least one heterologous gene in at least one of its cells.

The terms “transformants” or “transformed cells” include the primary transformed cell and cultures derived from that cell without regard to the number of transfers. All progeny may not be precisely identical in DNA content, due to deliberate or inadvertent mutations. Mutant progeny that have the same functionality as screened for in the originally transformed cell are included in the definition of transformants.

Transformation of a cell may be stable or transient. The term “transient transformation” or “transiently transformed” refers to the introduction of one or more exogenous polynucleotides into a cell in the absence of integration of the exogenous polynucleotide into the host cell's genome. Transient transformation may be detected by, for example, enzyme-linked immunosorbent assay (ELISA), which detects the presence of a polypeptide encoded by one or more of the exogenous polynucleotides. Alternatively, transient transformation may be detected by detecting the activity of the protein (e.g. β-glucuronidase) encoded by the exogenous polynucleotide. The term “transient transformant” refers to a cell which has transiently incorporated one or more exogenous polynucleotides. In contrast, the term “stable transformation” or “stably transformed” refers to the introduction and integration of one or more exogenous polynucleotides into the genome of a cell. Stable transformation of a cell may be detected by Southern blot hybridization of genomic DNA of the cell with nucleic acid sequences which are capable of binding to one or more of the exogenous polynucleotides. Alternatively, stable transformation of a cell may also be detected by enzyme activity of an integrated gene in growing tissue or by the polymerase chain reaction of genomic DNA of the cell to amplify exogenous polynucleotide sequences. The term “stable transformant” refers to a cell which has stably integrated one or more exogenous polynucleotides into the genomic or organellar DNA (chloroplast and/or mitochondria). It is to be understood that a plant or a plant cell transformed with the nucleic acids, constructs and/or vectors of the present invention can be transiently as well as stably transformed.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

### PREFERRED MODES FOR CARRYING OUT THE INVENTION

The present invention provides plants with enhanced tolerance to diseases caused by a variety of insect-transmitted viruses.

The strategy employed to develop the plants of the present invention is based on a novel concept, taking advantage of the fact that some, and perhaps all, plant viruses transmitted by insect vectors in a circulative manner, while being in the insect haemolymph, interact with GroEL homologues produced by the vector endosymbiotic bacteria. Binding between GroEL and virions occurs in vivo in the transmitting insect and in vitro. It has been suggested that GroEL-virus interaction could be a mechanism shared by plant circulative viruses to avoid destruction in the haemolymph.

Hitherto, the methods and means for providing virus-resistance plants were based on the pathogen-derived resistance concept, which involve the expression of functional as well as dysfunctional pathogen genes such as coat protein, replicase, and movement protein by the plant. RNA-mediated virus resistance proved to be more efficient than protein-mediated resistance, but was shown to be highly-sequence dependent and therefore with a narrower-range potential. It was shown to trigger existing plant antiviral mechanisms leading to the degradation of viral RNA, and was collectively named RNA-mediated gene silencing. It was found that some viruses establish themselves by expressing viral proteins which interfere with plant host silencing mechanisms. Transgenic plants have been developed which exploited the mechanism of silencing via double-stranded RNA sequences. However, transgenic RNA-mediated resistance can be overcome by strong silencing suppressors of non-related viruses in mixed infections. Recent strategies have targeted the formation of infectious virions by expressing peptides in transgenic plants which interfere with homo-multimerization of the coat protein. Some of these strategies have been used to obtain TYLCV-resistant tomato plants.

In contrary to the hitherto known methods of breeding for resistant plants, the present invention utilizes the general phenomenon of GroEL-virus binding to generate transgenic plants expressing GroEL, particularly expressing GroEL in their phloem, which are resistant to a variety of insect-transmitted viruses. Once inoculated by their vector, phloem-limited circulative viruses are trapped by GroEL in the plant phloem, such that invasion of phloem-associated cells and long distance movement is significantly inhibited, rendering the plants resistant to the viruses.

Thus, according to one aspect, the present invention provides a transgenic plant comprising at least one cell transformed with a polynucleotide encoding GroEL protein, or a plant-virus binding fragment, variant or homologue thereof. According to certain embodiments, the transgenic plant has an enhanced tolerance to at least one disease caused by an insect-transmitted plant pathogenic virus compared to a non-transgenic plant.

The concept of the present invention has been exemplified by expressing a gene encoding a GroEL homologue, particularly GroEL homologue from the endosymbiotic bacteria of the whitefly B. tabaci in transgenic tomato or tobacco plants and testing these plants following whitefly-mediated inoculation of TYLCV or CMV. The viruses capable of binding GroEL have a capsid protein with a basic isoelectric point and are rich in arginine residues; they also have a globular (or geminate) shape. Four viruses belonging to four different taxonomic groups have been examined: one virus has a DNA genome (TYLCV) and the other three, CMV, grapevine virus A (GVA) and tobacco mosaic virus (TMV) have an RNA genome. TYLCV and CMV have geminate or globular shapes, while TMV and GVA are filamentous.

Plants, as well as other eukaryotic organisms express hear shock proteins (Hsps) whose expression is increased when the cells are exposed to elevated temperatures or other stress. Heat shock proteins belong to the chaperonin family, and Hsp 60 share high homology with bacterial GroEL. Thus, the present invention explicitly encompasses transgenic plants transformed with heterologous as well as endogenous polynucleotide encoding GroEL and GroEL homologues, as long as the encoded protein is capable of binding a plant pathogenic virus.

### Producing the Transgenic Plants

Cloning of a polynucleotide encoding a GroEL protein can be performed by any method as is known to a person skilled in the art. Various DNA constructs may be used to express GroEL protein homologue in a desired plant.

The present invention provides a DNA construct or an expression vector comprising a polynucleotide encoding a GroEL homologue, which may further comprise a plant promoter.

A number of promoters which are active in plant cells have been described in the literature. These include the nopaline synthase (NOS) promoter (Ebert P R et al., Proc. Natl. Acad. Sci. U.S.A. 84: 5745-5749, 1987), the octopine synthase (OCS) promoter, the caulimovirus promoters such as the cauliflower mosaic virus (CaMV) 19S promoter (Lawton M A et al., Plant Mol Biol 9: 315-324, 1987) and the CaMV 35S promoter (Odell J T et al., Nature 313: 810-812, 1985), the figwort mosaic virus 35S-promoter; the light-inducible promoter from the small subunit of ribulose-1,5-bis-phosphate carboxylase (ssRUBISCO), the Adh promoter (Walker J C et al., 1987 Proc Natl Acad Sci USA 84: 6624-6628), the sucrose synthase promoter (Yang N S et al., 1990 Proc Natl Acad Sci USA. 87: 4144-4148), the R gene complex promoter (Chandler V L et al., 1989 The Plant Cell 1: 1175-1183), and the chlorophyll_{αβ} binding protein gene promoter, and the like. These promoters have been used to create DNA constructs which have been expressed in plants.

For the purpose of expression in certain tissues of the plant, such as the leaf, seed, root or stem, and particularly the phloem, it is preferred that the promoters utilized according to the teachings of the present invention have relatively high expression in these specific tissues. For this purpose, one may choose from a number of promoters for genes with tissue- or cell-specific or -enhanced expression. According to certain embodiments, the present invention utilizes an Arabidopsis phloem-specific promoter.

The DNA constructs or vectors may also include with the coding region of interest a nucleic acid sequence that acts, in whole or in part, to terminate transcription of that region. For example, such sequences have been isolated including the Tr7 3′ sequence and the NOS 3′ sequence (Ingelbrecht I L W et al., 1989 The Plant Cell 1: 671-680; Bevan M, et al., 1983 Nucleic Acids Res 11: 369-385), or the like.

The DNA constructs or vectors may also include a selectable marker. Selectable markers may be used to select for plants or plant cells that contain the exogenous genetic material. Examples of such include a neo gene (Potrykus I et al., 1985 Mol. Gen. Genet 199: 183-188) which codes for kanamycin resistance, such that cells/plants expressing same can grow on a kanamycin-containing medium; a bar gene which codes for bialaphos resistance; a mutant EPSP synthase gene which encodes glyphosate resistance; a nitrilase gene which confers resistance to bromoxynil (Stalker D M et al., 1988 J Biol Chem 263: 6310-6314); and a methotrexate resistant DHFR gene (Thillet J et al., 1988 J Biol. Chem. 263: 12500-12508).

The DNA construct or expression vector may also include translational enhancers. DNA constructs could contain one or more 5′ non-translated leader sequences which may serve to enhance expression of the gene products from the resulting mRNA transcripts. Such sequences may be obtained from viral RNAs, from suitable eukaryotic genes, or from a synthetic gene sequence, and the like.

The DNA construct of the present invention can be utilized to stably or transiently transform plant cells. In stable transformation, the nucleic acid molecule is integrated into the plant genome, and as such it represents a stable and inherited trait. In transient transformation, the nucleic acid molecule is expressed by the cell transformed but is not integrated into the genome, and as such represents a transient trait.

The principal methods of the stable integration of exogenous DNA into plant genomic DNA include: (i) Agrobacterium-mediated gene transfer (see for example Klee H et al., 1987 Annu Rev Plant Physiol 38: 467-486; and Gatenby A A, 1989 Plant Biotechnology, pp. 93-112 S Kung and C J Arntzen, Eds., Butterworth Publishers, Boston, Mass.); and (ii) Direct DNA transfer include microinjection, electroporation, and microprojectile bombardment (U.S. Pat. No. 6,723,897 and references therein, which is incorporated by reference as if fully set forth herein).

Agrobacterium-mediated transfer is a widely applicable system for introducing genes into plant cells because the DNA can be introduced into whole plant tissues, thereby bypassing the need for regeneration of an intact plant from a protoplast. The Agrobacterium-mediated system includes the use of plasmid vectors that contain defined DNA segments which integrate into the plant genomic DNA. Methods of inoculation of the plant tissue vary depending upon the plant species and the Agrobacterium delivery system. A widely used approach is the leaf-disc procedure, which can be performed with any tissue explant that provides a good source for initiation of whole-plant differentiation. A supplementary approach employs the Agrobacterium delivery system in combination with vacuum infiltration. The Agrobacterium system is especially useful for in the creation of transgenic dicotyledonous plants.

In electroporation, the protoplasts are briefly exposed to a strong electric field, opening up mini-pores to allow DNA to enter. In microinjection, the DNA is mechanically injected directly into the cells using micropipettes. In microparticle bombardment, the DNA is adsorbed on microprojectiles such as gold or tungsten particles, and the microprojectiles are physically accelerated into cells or plant tissues.

Transient transformation can be effected by any of the direct DNA transfer methods described above or by viral infection using modified plant viruses.

Viruses that have been shown to be useful for the transformation of plant hosts include cauliflower mosaic virus (CaMV), tobacco mosaic virus (TMV), and baculovirus (BV). Transformation of plants using plant viruses is described in, for example, Communications in Molecular Biology: Viral Vectors, Cold Spring Harbor Laboratory, New York, pp. 172-189.

Construction of plant RNA viruses for the introduction and expression of non-viral exogenous nucleic acid sequences in plants is known in the art and demonstrated by the above references as well as by Dawson W O et al., 1989 Virology 172: 285-292.

If the transforming virus is a DNA virus, one skilled in the art may make suitable modifications to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of the DNA will produce the coat protein, which will encapsidate the viral DNA. If the virus is an RNA virus, the virus is generally cloned as a cDNA and inserted into a plasmid. The plasmid is then used to make all of the plant genetic constructs. The RNA virus is then transcribed from the viral sequence of the plasmid, followed by translation of the viral genes to produce the coat proteins which encapsidate the viral RNA.

Transformation of plant protoplasts has been reported using methods based on calcium phosphate precipitation, polyethylene glycol treatment, electroporation, and combinations of these treatments (see, for example, Marcotte W R et al., Nature 335: 454-457, 1988).

The regeneration, development, and cultivation of plants from single plant protoplast transformants or from various transformed explants is well known in the art This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

Following stable transformation, plant propagation then occurs. The most common method of plant propagation is by seed. The disadvantage of regeneration by seed propagation, however, is the lack of uniformity in the crop due to heterozygosity, since seeds are produced by plants according to the genetic variances governed by Mendelian rules. In other words, each seed is genetically different and each will grow with its own specific traits. Therefore, it is preferred that the regeneration be effected such that the regenerated plant has identical traits and characteristics to those of the parent transgenic plant. The preferred method of regenerating a transformed plant is by micropropagation, which provides a rapid, consistent reproduction of the transformed plants.

Micropropagation is a process of growing second-generation plants from a single tissue sample excised from a selected parent plant or cultivar. This process permits the mass reproduction of plants having the preferred tissue and expressing a fusion protein. The newly generated plants are genetically identical to, and have all of the characteristics of, the original plant. Micropropagation allows for mass production of quality plant material in a short period of time and offers a rapid multiplication of selected cultivars with preservation of the characteristics of the original transgenic or transformed plant. The advantages of this method of plant cloning include the speed of plant multiplication and the quality and uniformity of the plants produced.

Micropropagation is a multi-stage procedure that requires alteration of culture medium or growth conditions between stages. The micropropagation process involves four basic stages: stage one, initial tissue culturing; stage two, tissue culture multiplication; stage three, differentiation and plant formation; and stage four, greenhouse culturing and hardening. During stage one, the tissue culture is established and certified contaminant-free. During stage two, the initial tissue culture is multiplied until a sufficient number of tissue samples are produced to meet production goals. During stage three, the newly grown tissue samples are divided and grown into individual plantlets. At stage four, the transformed plantlets are transferred to a greenhouse for hardening where the plants' tolerance to light is gradually increased so that they can continue to grow in the natural environment.

Additionally or alternatively, since the transgenic plants regenerated from tissue culture (“T₀” generation) are usually heterozygous for the desired trait, the plants are selfed and their progeny (“T₁” generation) is tested for homozygosity. Production of homozygous plants that do not segregate for the desired trait or gene, may requires 2-4 cycles of progeny selfing.

As non-limiting examples, the present invention discloses the production of transgenic tomato and tobacco plants expressing the whitefly GroEL protein, having an increased tolerance to GroEL binding viruses.

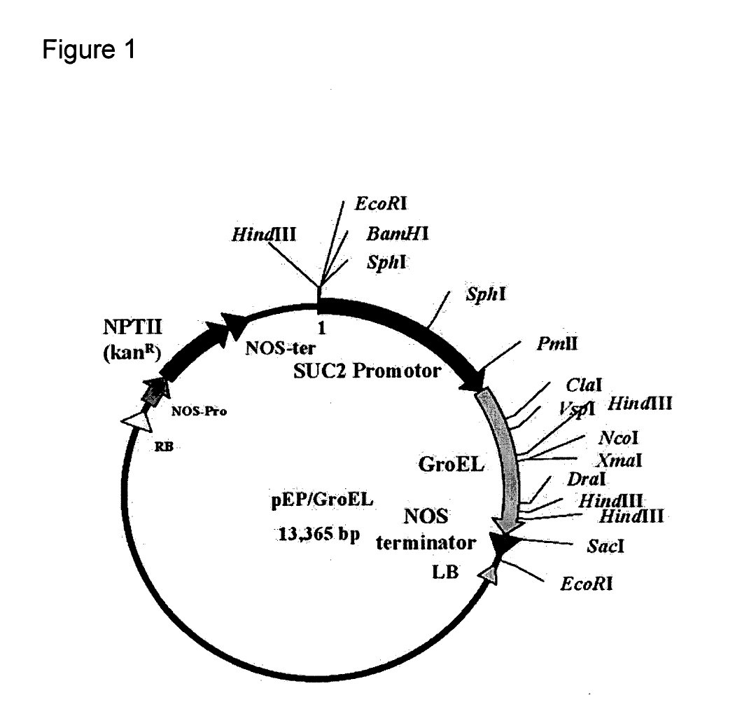

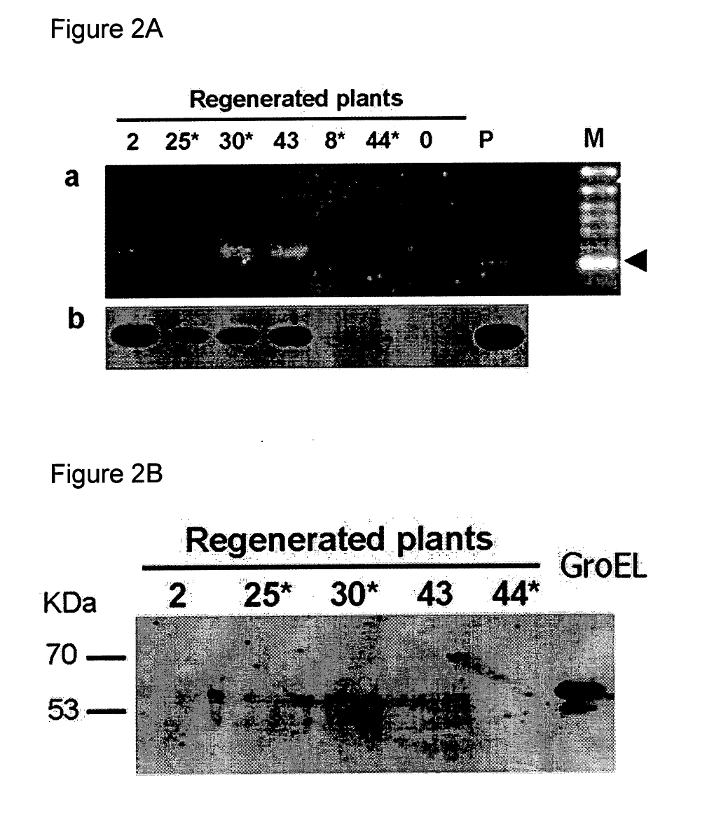

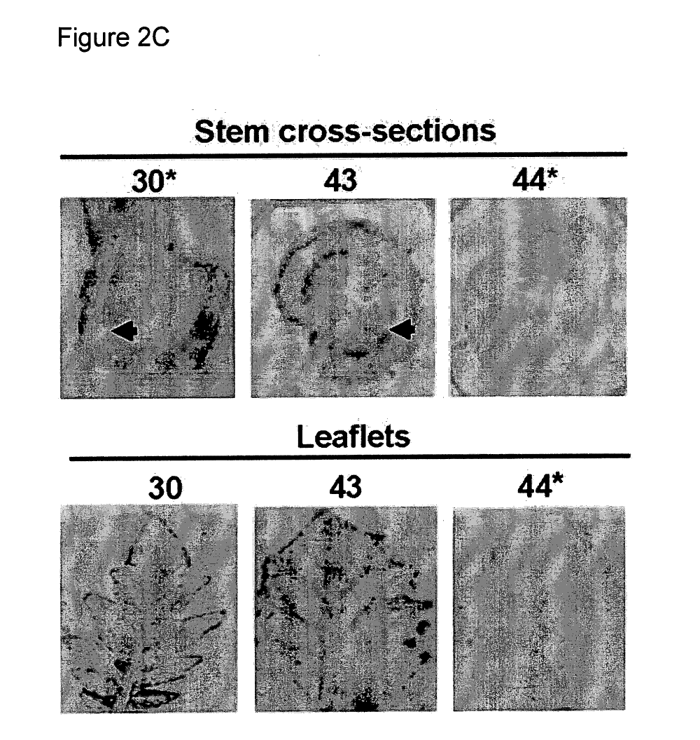

According to certain embodiments, a whitefly GroEL gene (SEQ ID NO:1) was cloned in an Agrobacterium binary vector under the control of an Arabidopsis phloem-specific promoter (FIG. 1). In Arabidopsis leaves, this promoter regulates the expression of the companion cell-specific AtSUC2 Suc-H⁺ symporter gene (Truernit E and Sauer N, 1995 Planta 196:564-570). The tissue-specificity of the promoter has been demonstrated in tobacco (Wright K M et al., 2003, Plant Physiol. 131:1555-1565) but not in tomato. Immunodetection of GroEL indicated that the Arabidopsis promoter drove the expression of this protein in the tomato phloem as well (FIG. 2). The GroEL gene is of prokaryote origin and the question of its efficient expression in a eukaryotic environment has been raised. Examination of codon usage (Graphic Codon Usage Analyser http://www.gcua.de) indicated that the codon usage of the GroEL gene from B. tabaci fits for expression in tomato plants. Indeed GroEL was detected by Western blot analysis and was immunolocalized in the vascular system of the transgenic plants. The plants expressing the insect GroEL did not show any obvious phenotype or malformation.

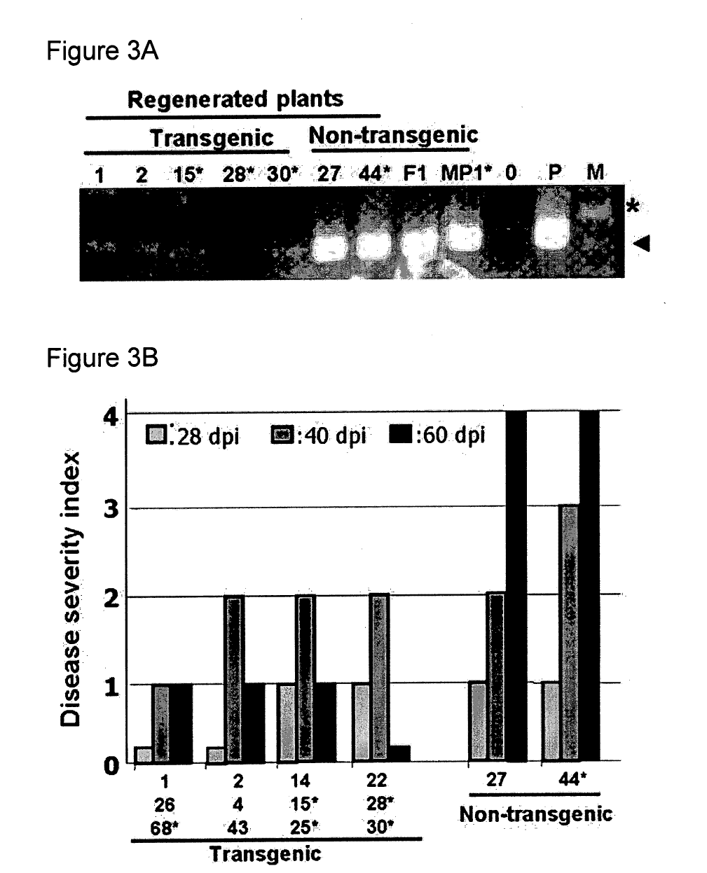

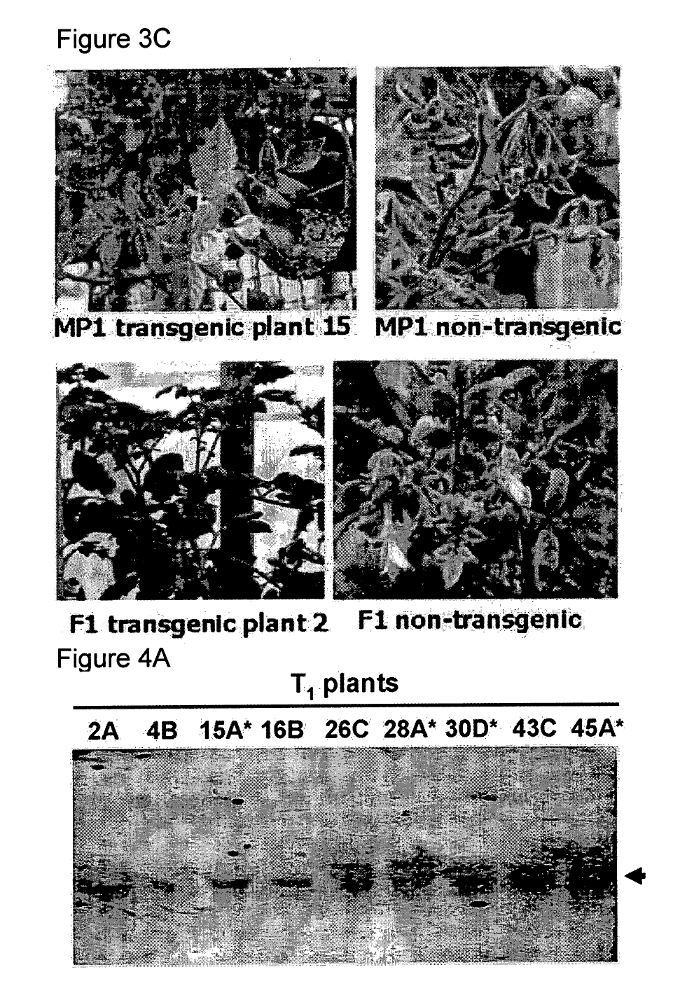

GroEL was expressed in the phloem of transgenic tomato plants issued from two different genotypes (FIG. 2), which presented excellent transformation and regeneration rates: an interspecific hybrid of Solanum pennellii×S. lycopersicum (Kunik T et al., 1994 BioTechnology 12: 500-504) and the tomato line MP1 (Barg R et al., 1997. J Exp Botany 48: 1919-1923). Results obtained with the hybrid and with MP1 were similar. MP1 was described as tolerant to TYLCV; however under the harsh inoculation conditions employed in the present invention, this line was as susceptible as susceptible cultivars such as FA144 (cv. Daniella). The GroEL-transgenic tomato plants exhibited good levels of resistance to whitefly-mediated inoculation of TYLCV, exhibiting very mild or no symptoms (FIG. 3). The transgenic progeny of the resistant plants were as resistant as their parents.

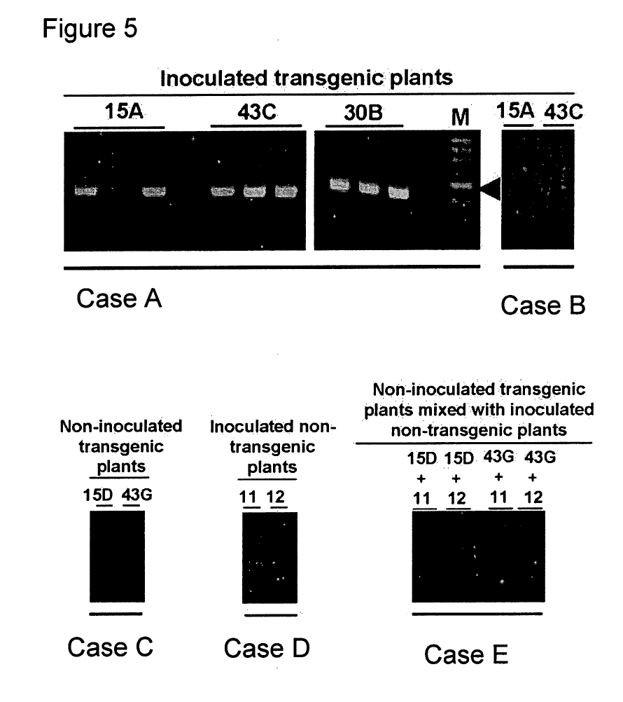

In “T₁” and “T₂” resistant plants, in vitro assays indicated that viral particles were bound to GroEL in the plant sap (FIG. 5). In all plants tested there was a strict correlation between the detection of GroEL-TYLCV complexes and the symptomless phenotype presented by the transgenic plants upon inoculation. These complexes were pre-formed; GroEL-TYLCV complexes were not detected when mixes of saps of non-infected transgenic plants and infected non-transgenic plants were incubated together in anti-GroEL coated tubes, probably because of the low concentration of free GroEL and free virus in the plant saps.

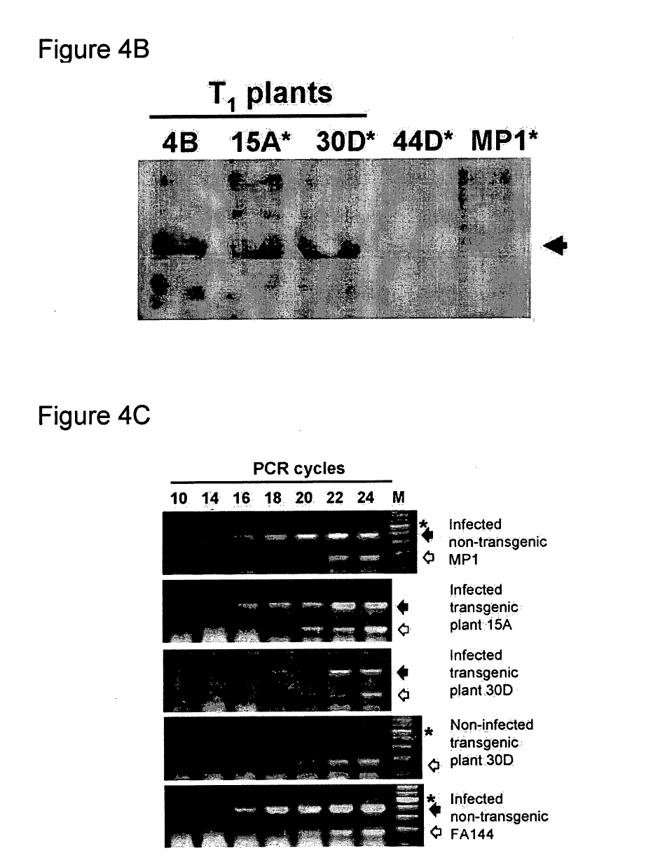

Without wishing to be bound by any specific mechanism or theory, the present invention is based in part on the paradigm that interaction between GroEL and TYLCV in the phloem may prevent invasion of phloem-associated cells, a first step necessary for the virus to replicate. PCR analyses showed that 2-3 weeks after inoculation the resistant transgenic tomato plants contained fewer viral DNA than susceptible non-transgenic plants; however, 6 weeks later the viral DNA amounts were similar in the symptomless transgenic plants and in the symptomatic non-transgenic plants (FIGS. 3 and 4). The pattern of viral DNA accumulation in the GroEL-expressing plants suggests that the virus replicates in these plants. It also implies that not all particles are immediately trapped by the chaperonins upon inoculation. Some virions seem to escape and invade companion cells where they replicate. It is also possible that the virus replicates after GroEL-virion complexes have penetrated these cells, and have dissociated (it is unlikely that complexes penetrate the cell nucleus). The fact that strong symptoms do not develop in infected transgenic plants indicates that the accumulation of virions is slow enough not to interfere with the growth of the plant. It is also possible that GroEL disturbs interactions between viral and plant proteins at one or more of the steps of the virus cycle: entry into the cell nucleus, exit into the cytoplasm, cell-to-cell and long distance movement.

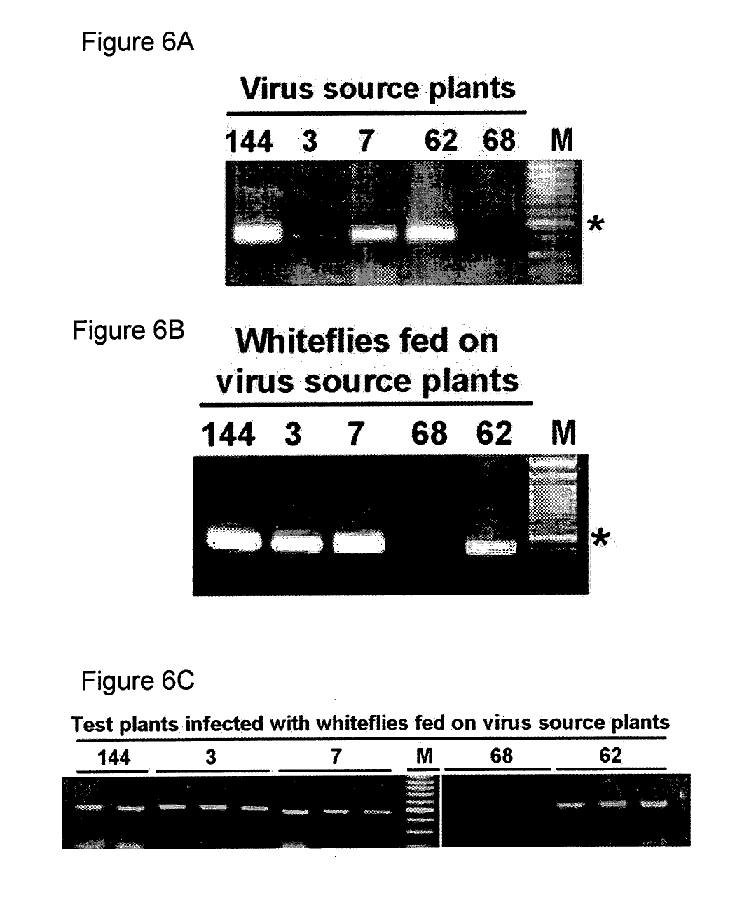

In the field, infected resistant tomato plants (resistance obtained by classical breeding) may serve as virus inoculum as do infected susceptible plants. Efficacy and rates of whitefly-mediated transmission of the TYLCV disease to susceptible plants is similar whether the virus source is from susceptible or from resistant plants, especially in the later stages of infection (Lapidot M and Friedmann M, 2002 Ann Appl Biol 140: 109-127). In the present invention it was examined whether whitefly-mediated transmission of virus from the infected transgenic plants was impacted by GroEL. It was found that infected GroEL-expressing resistant plants could serve as good a source of virus for whitefly-mediated transmission as did infected non-transgenic plants (FIG. 6). It was not identified whether whiteflies selectively ingurgitate free particles from the transgenic tomatoes, or ingurgitate GroEL-virus complexes as well. There might be enough free virions in the transgenic plant sap to be picked up by the insect stylets and transmitted to test plants, but not enough to induce a systemic infection. Alternatively, GroEL-virus complexes may be acquired during feeding and dissociate at some point in the insect digestive tract.

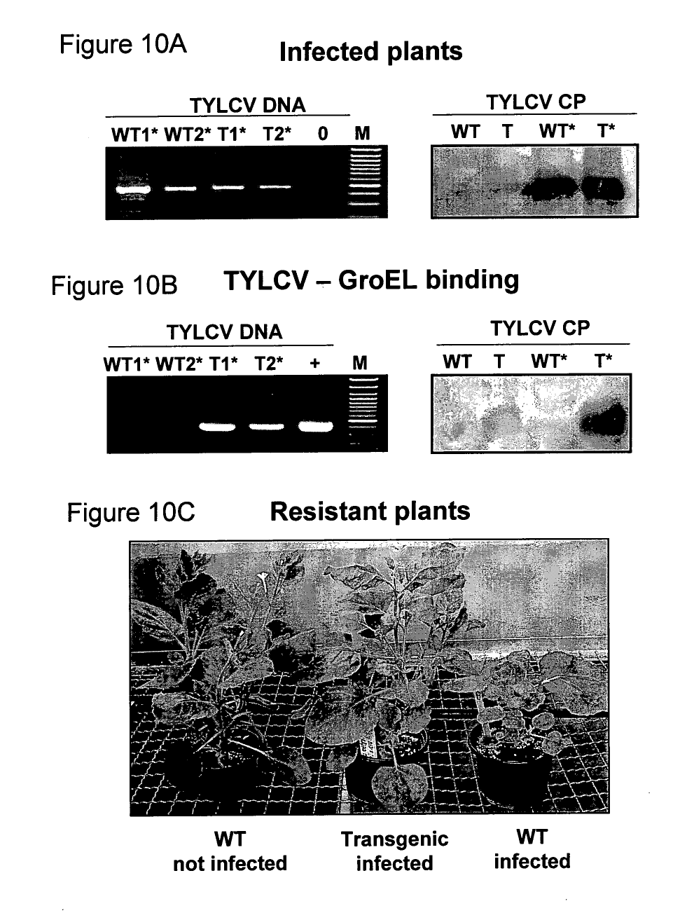

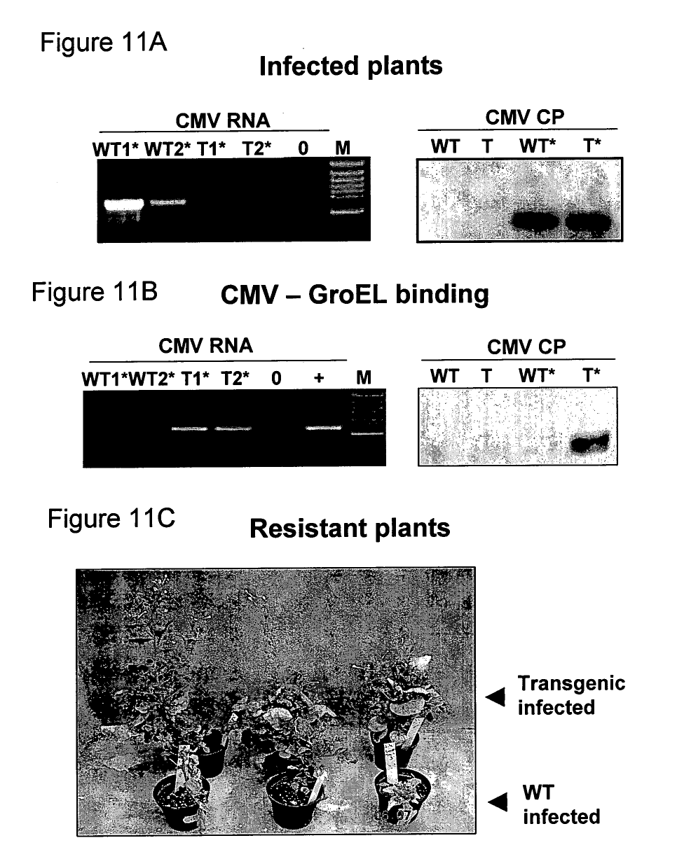

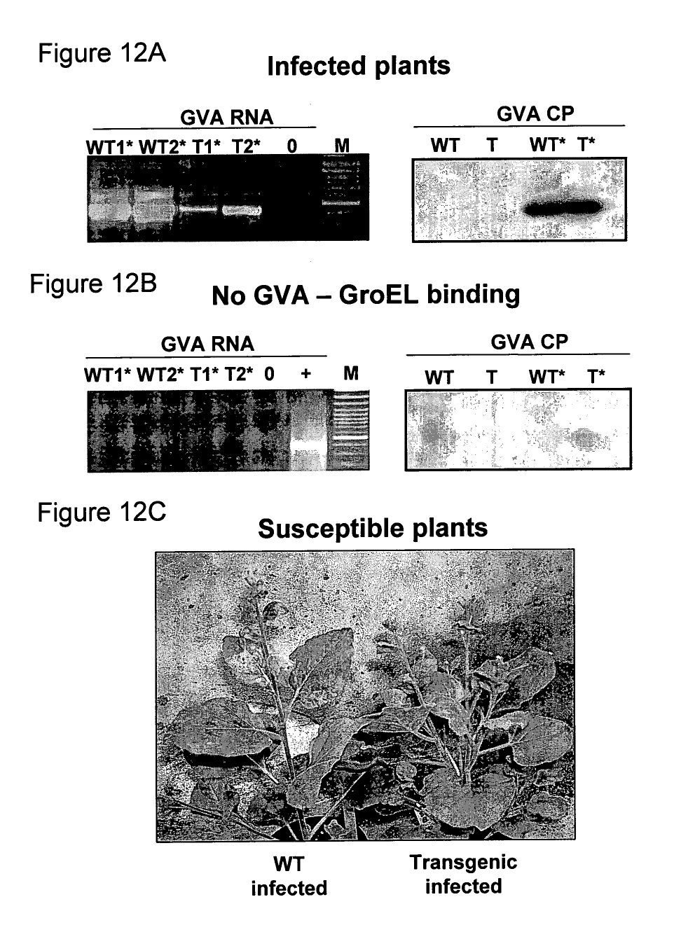

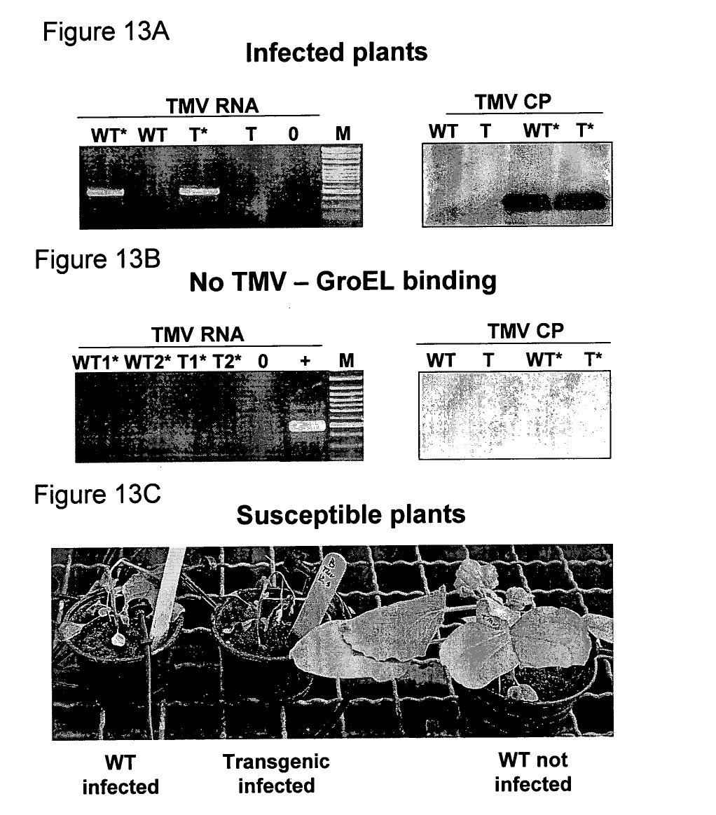

According to other embodiments, the whitefly GroEL gene was cloned in the Agrobacterium binary vector under the control of the constitutive 35S CaMV promoter. This system was used to produce transgenic tobacco plants (Nicotiana benthamiana) and examine their resistance to four insect-transmitted viruses: two that are expected to bind GroEL (TYLCV and CMV) and two that are not GroEL-binding viruses (TMV and GVA). The present invention now shows that indeed, N. benthamiana plants expressing the B. tabaci GroEL homologue were tolerant to the GroEL binding viruses TYLCV and CMV (FIGS. 10 and 11). Although the plants contained detectable amounts of virus, they presented either mild or no symptoms. GroEL-virus complex were detected in their sap. By comparison GVA and TMV, two viruses that do not bind to GroEL in vitro, produced strong disease symptoms in infected N. benthamiana plants, whether expressing the whitefly GroEL or not (FIGS. 12 and 13). GroEL-GVA/TMV complexes could not be detected in the sap of the transgenic tobacco.

### Engrafted Plants

According to another aspect, the present invention provides a plant comprising a transgenic rootstock comprising at least one cell transformed with a polynucleotide encoding GroEL protein, or a plant-virus binding fragment, variant or homologue thereof, and a non-transgenic graft, wherein the whole engrafted plant has an enhanced tolerance to at least one disease caused by an insect-transmitted plant pathogenic virus.

Grafting involves combining two independent plant parts into one plant. Such combination may be performed in various ways, including, but not limited to whip and tongue graft, splice graft, tip-cleft graft, side graft, saddle graft and bud graft (for further details see Garner R. J., The Grafter's Handbook, 5th Ed edition (March 1993) Cassell Academic; ISBN: 0304342742).

According to certain embodiments, the rootstock comprises a polynucleotide encoding Bemisia tabaci GroEL protein. According to certain typical embodiments, the GroEL protein has an amino acid sequence as set forth is SEQ ID NO:2 encoded by a polynucleotide comprising a nucleic acid sequence as set forth in SEQ ID NO:1.

As described hereinabove, GroEL expressing plants are resistant to viral diseases caused by insect-transmitted pathogenic viruses. The present invention now shows that the sap of susceptible, non-transgenic plants grafted onto a transgenic GroEL expressing rootstock contains GroEL protein homologue as well as GroEL-virion complexes. Without wishing to be bound by a specific mechanism or theory, the GroEL expressed in the transgenic rootstock may be translocated to the graft, wherein the GroEL present in the graft sap is capable of trapping the infectious virus. It should be noted, however, that the amount of GroEL protein in the graft should be such that it would suffice to confer resistance to the susceptible graft.

The following non-limiting examples hereinbelow describe the means and methods for producing the transgenic plants of the present invention. Unless stated otherwise in the Examples, all recombinant DNA and RNA techniques, as well as horticultural methods, are carried out according to standard protocols as known to a person with an ordinary skill in the art.

### EXAMPLES

### Material and Methods

### Sources of Virus, Insects, Plants and Antibodies

Bemisia tabaci of the B biotype (Cohen S, 1993 Phytoparasitica 21: 174) was reared on cotton plants (Gossypium hirusutum cv. Akala) grown in insect-proof wooden cages at 24-27° C., as previously described (Zeidan M and Czosnek H, 1991. J Gen Virol 72: 2607-2614).

An isolate of Tomato yellow leaf curl virus (TYLCV) from Israel (Navot N et al., 1991 Virology 185: 151-161) was maintained in tomato plants (Solanum lycopersicum cv. Daniella FA144) by whitefly-mediated transmission. Cucumber mosaic virus (CMV), Fny-CMV RNA-3 (D10538), was maintained in melon (Cucumis melo, cv. Halles best Jumbo). Grapevine virus A (GVA) was maintained on N. benthamiana (X75433). Purified Tobacco mosaic virus (TMV) particles (X68110) were a gift of Prof I. Sela (The Hebrew University of Jerusalem, Rehovot, Israel).

All the antibodies used in this study were polyclonal. Antibodies to native Buchnera GroEL from Myzus persicae raised in rabbits were a gift of Dr J F J M van den Heuvel (IPO-DLO, Wageningen, The Netherlands). Antibodies against TMV were a gift of Prof A. Gera (Agricultural Research Organization, Bet Dagan, Israel). Anti-GroEL and anti-TMV antibodies were cleaned on a protein A-sepharose CL-4B column (Sigma). Antibodies against CMV were purchased from Bioreba (Reinach, Switzerland). Antibodies against TYLCV were a gift of Dr. F. Akad (University of Florida, USA). Antibodies against GVA were a gift of Dr. M. Mawassi (Agricultural Research Organization, Bet Dagan, Israel)

The tomato line MP1 (Barg R et al., 1997, ibid) and an interspecific hybrid of S. pennellii×S. lycopersicum (Kunik T et al., 1994. BioTechnology: 12:500-504), named in short F1, were used for transformation. Grafts were done in a specialized commercial nursery (Hishtil, Ashkelon, Israel). Grafting was performed just below the cotyledons; hence scions did not include leaves.

Cloning of the GroEL Gene from B. tabaci Under the Promoter of the Phloem-Specific Sucrose Transporter Gene SUC2 from Arabidopsis thaliana

The gene encoding the GroEL homologue from secondary endosymbiotic bacteria of B. tabaci (accession number AF130421) was cloned under the control of the promoter of the Arabidopsis thaliana SUC2 sucrose-H⁺ transporter gene, AtSUC2 (accession number X79702), a phloem-specific gene (Truernit E and Sauer N, 1995 Planta 196: 564-570). The insect full-length GroEL gene (Morin S et al., 2000, ibid) was isolated by PCR using the forward primer: 5′-TTTCATGACAGCTAAAGACTTAAAATTTGG-3′ (SEQ ID NO:3) which contains the AUG translation initiation codon as part of an added RcaI site (changes to obtain the RcaI site are underlined), and the reverse complement primer: 5′-TGAGCTCTTACATCATACCATTCATTCCGCC-3′ (SEQ ID NO:4), which contains an added Sad site (the added Sad is underlined). The PCR cycling consisted of a 4 min initial denaturation at 94° C., annealing of 2 min at 54° C., and elongation of 3 min at 72° C., followed by 25 cycles of 94° C. for 30 sec, 54° C. for 1 min, 72° C. for 2 min, and a final step at 72° C. for 15 min. The amplified 1,665 by GroEL gene was initially cloned into pGEM-T Easy (Promega, Madison, USA) following RcaI/SacI cleavage. The RcaI/SacI fragment was further sub-cloned into the corresponding restriction sites of plasmid pUC19-AtSUC-mpCMV-GFP (Stadler R et al., 2005 Plant J 41:319-331), which contains the movement protein gene of Cucumber mosaic virus fused to GFP (mpCMV-GFP) under the control of the Arabidopsis phloem-specific promoter of the SUC2 gene (a gift of Dr S. Wolf). As a result, GroEL which replaced the mpCMV-GFP was under the control of the SUC2 promoter. The cassette containing the GroEL gene and the SUC2 promoter was cleaved by PstI/SacI and inserted into the SdaI/SacI sites (PstI and SdaI have compatible ends) of the Agrobacterium tumefaciens binary vector pBI121 (Accession number AF485783), replacing the 35S CaMV promoter and the GUS gene (FIG. 1). The binary plasmid (named pEP/GroEL) was mobilized into Agrobacterium LBA4404 by electroporation.

Cloning of the GroEL Gene Under the 35S CaMV Promoter into Agrobacterium Binary Vector

Bemisia tabaci B biotype was kindly provided by the Plant Entomology Laboratory, Plant Protection Division, Shizuoka Agricultural Experiment Station, Iwata, Japan. B. tabaci was reared on tomato plants (Solanum lycopersicum) grown in insect-proof wooden cages at 25° C. Total DNA from approximately 1,000 whiteflies (4 to 7 days after emergence) was isolated using the QIAamp tissue kit protocol for insects (Qiaqgen, Chatsworth, Calif.). Initially, the full length B. tabaci GroEL gene was PCR-amplified using the above identified forward and reverse primers having SEQ ID NO:3 and SEQ ID NO:4 respectively, cloned using the pGEM-T Easy vector system (Promega) as described (Pascal E et al., 1993 Plant cell 5: 795-807). After sequencing, two primers with an XbaI site and an SalI site, GroEL-XbaI (5′-TCTAGAATGGCAGCTAAAGAC-3′, SEQ ID NO:5) and GroEL-SalI (5′-GAGCTCTTACATCATACCATTC-3′, SEQ ID NO:6), were designated to obtain a full-length clone (pTGrEL7). pTGrEL7 was digested with XbaI and Sad, and the GroEL fragment was ligated to the binary vector pBI121 digested with XbaI and Sad to produce the plasmid pBI121-GroEL. The resultant binary plasmid pBI121-GroEL, which contained the GroEL gene flanked by the promoter and terminator, was introduced into Agrobacterium LBA4404 by the triparental mating method.

### Transformation and Regeneration of Tomato Plants

Surface sterilized tomato seeds (MP1 and F1 described hereinabove) were germinated on germination Nitsch medium (Nitsch J P and Nitsch C, 1969 Science 163: 85-87). Cotyledon and hypocotyls were excised from 10-day-old seedlings, discarding both extremities (leaving approximately ⅔ of the tissues), and cultured for 48 h on MS agar medium (Murashige T and Skoog F, 1962 Physiologia Plantarum 15: 473-479) in 9-cm-diameter Petri dishes. Agrobacterium containing the binary vector pEp/GroEL was grown for 16 h in YT medium containing 50 mg/l rifampicin and 50 mg/l kanamycin. The culture was diluted with MS medium to O.D.600 nm=0.3, and acetosyringone was added to a final concentration of 100 mM. Cotyledons and hypocotyls were submerged in the bacteria suspension for 2 h. The bacteria were removed and the explants were cultivated on MS agar plates for 48 h in the dark at 24° C. The explants were then transferred to selective regeneration medium (solidified MS salts with Nitsch vitamins (Nitsch and Nitsch, 1969, ibid), containing 400 mg/l carbenicillin, 70 mg/l kanamycin and 1 mg/l zeatin). Regenerated explants were transferred to fresh medium biweekly. Green shoots, 1-3 cm high, were separated from the original explants and transferred to Nitsch medium containing 150 mg/l carbenicillin, 50 mg/l kanamycin and 1 mg/l indolebutyric acid (IBA) for rooting. Rooted plants were transplanted to soil in 1 liter pots and kept in the greenhouse in controlled conditions fitting the regulations of the Israel Plant Protection Authorities.

Transformation of Nicotiana benthamiana Plants

Agrobacterium strain LBA4404 was used to transform leaf discs of N. benthamiana as described (Pascal et al., 1993, ibid). Selection for transformation was done on medium containing kanamycin (150 μg/ml). Kanamycin-resistant shoots were collected, placed on rooting medium, grown to height of 5 to 6 cm, and transferred to soil. T₀ lines were self-pollinated and T₁ seeds germinated on MS medium containing 50 μg/ml of kanamycin. T₁ seedlings were transplanted in soil one month after germination. Expression of GroEL gene in transformed plants was ascertained by Western blot analysis, using a polyclonal antibody to native Buchnera GroEL from Myzus persicae raised in rabbit (van den Heuvel J F J M, 1994, ibid). The plants were self pollinated and selected until they did not segregated and were homozygous for the GroEL and NptII genes.

### PCR-Detection of GroEL and NPTII DNA in Tomato Plants

Plants were assayed for the presence of the GroEL gene (accession number AF130421) by PCR using the sense primer GroEL-P751 5′-GCAGAAGATGTTGAAGGTGAAGC-3′ (SEQ ID NO:7, starting 751 nucleotides from the initiation codon), and the reverse complementary primer GroEL-P1229 5′-CCTTCTTCTACTGCTGCTTCTTGT-3′ (SEQ ID NO:8, nucleotides 1229-1206). PCR was performed as described above, amplifying an about 478 by GroEL DNA fragment. Plants were also analyzed by PCR for the presence of the NPTII gene (accession number AF485783) using primers NptII-F (SEQ ID NO:9, nucleotides 2874 to 2895 of the NPTII gene: 5′-GCCGCTTGGGTGGAGAGGCTAT-3′) and NptII-R (SEQ ID NO:10, nucleotides 2550 to 2539: 5′-GAGGAAGCGGTCAGCCCATTCG-3′).

### Detection of GroEL DNA by Southern Blot Hybridization

Thirty μg of tomato total genomic DNA extracted as described before (Bernatzky R and Tanksley S D, 1986, Mol Gen Gen 203: 8-14) were digested to completion with EcoRI. The DNA fragments were subjected to 0.8% agarose gel electrophoresis and transferred to Hybond N⁺ membranes (Amersham, UK). GroEL specific probes were prepared by random primer labeling with α³²P-dCTP. Hybridization was carried out at 65° C. for 18 h. The blot was washed with 0.1×SSC at 65° C. and exposed for 72 h at [−70]° C. using an intensifying screen and Kodak Biomax film.

### Western Blot Immunodetection of GroEL

Leaf protein extracts were prepared in sample buffer (Laemmli U K, 1970 Nature 227: 680-685) and subjected to 10% SDS-PAGE. After electrophoresis, proteins were electroblotted onto a Hybond-C Extra membrane (Amersham) at 4° C. for 2 h at 110V, using transfer buffer (25 mM Tris HCl pH 8.3-192 mM glycine) supplemented with 10% (v/v) methanol. The membranes were blocked for 1 h at 22-25° C. with 2% bovine serum albumin (BSA) in 10 mM Tris HCl pH 7.5-150 mM NaCl containing 0.1% Tween 20 (TBS-T). GroEL was immunodetected as described (Akad F et al., 2004, ibid). Briefly, membranes were incubated for 18 h at 4° C. with the GroEL antiserum (diluted 1:1000). All subsequent steps were done at 22-25° C. Following five washes of 10 min each in TBS-T, membranes were incubated for 1 h with horseradish peroxidase-linked anti-rabbit IgG. After intensive washes with TBS-T, immobilized conjugates were visualized by enhanced chemiluminescence (ECL, Amersham Life Science), followed by exposure to X-ray film.

### Immunodetection of GroEL in Tissue Prints

Tomato plant stems were cut and sections were stamped on Hybond-C Extra membranes (Amersham Life Science). Detached leaves were laid on membrane wetted with TBS-T and subjected to vacuum at ambient temperature for 1 h. The tissue prints (Akad F et al., 2004 ibid) were incubated with 2% BSA blocking solution for 1 h. GroEL was detected using the GroEL antibody as described above.

### Inoculation of Tomato and Tobacco Plants

TYLCV was inoculated as follows: An isolate of TYLCV from Israel was maintained in tomato plants (S. lycopersicum cv. Daniella FA144) by whitefly-mediated transmission. Tobacco plants (N benthamiana) were caged for 72 h with whiteflies (about 50 insects per plant) that acquired TYLCV from the infected tomato plants during a 72 h acquisition access period.

CMV was inoculated as follows: Young leaves from infected melon were grinded in 10 mM sodium phosphate buffer pH 7.2, 1 mM EDTA pH 8.0, 0.2% β-mercaptoethanol (2 ml per gram tissue). 100 μl of extract mixed with carborendum were used to inoculate two leaves per plant.

GVA was inoculated as follows: Extracts from infected N. benthamiana leaves were prepared and used as inoculum as described hereinabove for CMV.

TMV was inoculated as follows: Purified virions were mixed with carborendum in 10 mM sodium phosphate buffer, pH 7.2. 10 μg of virions in 100 μl 0.01 M phosphate buffer pH. 7.4 were used to inoculate two large leaves per plant.

In each experiment, twenty transgenic plants and ten non-transgenic plants were inoculated with each virus and analyzed for the presence of the virus, of virus-GroEL complexes and for symptoms. Four independent experiments were conducted.

The transgenic plants, whether inoculated or not, were grown in a greenhouse in controlled conditions with permission and according to the regulations of the Israel Ministry of Agriculture Plant Protection and Inspection Services.

### Semi-Quantitative PCR Analysis of TYLCV DNA in Transgenic Tomato Plants

A 100 μl reaction mixture was prepared, containing 100 ng of tomato total genomic DNA, 1 μl of a 25 mM mixture of the four dNTPs, 10 μl Taq polymerase buffer (×10), 1 unit of Taq DNA polymerase and 2.5 pmoles of two TYLCV-specific primers (accession number X15656): virion strand (position 61-80) 5′-ATACTTGGACACCTAATGGC-3′ (SEQ ID NO:11) and complementary strand (position 473-457) 5′-AGTCACGGGCCCTTACA-3′ (SEQ ID NO:12). Tomato β-actin served as positive control; 2.5 pmoles of two β-actin-specific primers were used (accession number BT013524): sense (position 772-791) 5′-GGAAAAGCTTGCCTATGTGG-3′ (SEQ ID NO:13), and complementary sense (position 951-932) 5′-CCTGCAGCTTCCATACCAAT-3′ (SEQ ID NO:14). The mixture was aliquoted in 10 tubes and subjected to PCR. Cycling was as follows: initial denaturation at 95° C. for 3 min followed by cycles of 30 sec at 95° C., 30 sec at 55° C. and 1 min at 72° C. The reaction was stopped after various numbers of cycles and 5 μl of the PCR products were subjected to electrophoresis in 1% agarose gel, in Tris-Phosphate-EDTA buffer (TAE) and stained with ethidium bromide (0.5 μg/ml).

### Virus Detection by PCR and Reverse-Transcription PCR (RT-PCR) in Tobacco Plants

TYLCV was detected using primers derived from the virus coat protein gene TYLCVcp sense (nucleotides 530-548) 5′ GAAGGCTGAACTTCGACAG 3′ (SEQ ID NO:15) and TYLCVcp reverse sense (nucleotides 928-908) 5′ ATTGGGCTGTTTCCATAG GGC 3′ (SEQ ID NO:16); the PCR product is 411 base-pair long. CMV was detected using primers derived from the virus movement protein CMVmp sense (nucleotides 148-170) 5′ TAACTCAACAGTCCTCAGCGGC 3′ (SEQ ID NO:17) and CMVmp reverse sense (nucleotides 744-722) 5′ CTGACGGTTTTGTTTGCTCAGC 3′ (SEQ ID NO:18); the PCR product is 596 base-pair long. GVA was detected using primers derived from the virus V1 gene GVAv1 sense (nucleotides 6410-6428) 5′ GACAAATGGCACACTACG 3′ (SEQ ID NO:19) and GVAc1 complementary sense (nucleotides 6840-6818) 5′AAGCCTGACCTAGTCATCTTG G3′ (SEQ ID NO:20); the PCR product is 430 base-pair long. TMV was detected using primers derived from the virus coat protein gene TMVcp-sense (nucleotides 5712-5738) 5′ ATGTCTTACAGTATCACTACTCCATAT 3′ (SEQ ID NO:21) and TMVcp-reverse sense (nucleotides 6190-6168) 5′ CAAGTTGCAGGACCAGAGGTCCA 3′ (SEQ ID NO:22); the PCR product is 472 base-pair long.

### Capture of TYLCV Using Anti-GroEL Coated Tubes and Detection by PCR

PCR tubes were filled with anti-GroEL antibody diluted (1:1000) in ELISA coating buffer (15 mM Na₂CO₃, 35 mM NaHCO₃ and 0.02% NaN₃, pH 9.6), incubated for 3 h at 37° C., followed by incubation with 2% BSA in 10 mM Tris HCl pH 7.5-150 mM NaCl containing 0.1% Tween 20 (TBST) for 1 h at 37° C., and washed four times with TBST (5 minutes each wash). 100 μl of homogenates of cleared sap from tomato stems and 200 μl from N. benthamiana homogenates (1 ml/0.5 g tissue) were added to the anti-GroEL antibody coated support, incubated for either 1 h at 37° C. or 18 h at 4° C., and washed four times with TBST. For detection of TYLCV, PCR reagents were added directly to the tube with the TYLCV-specific primers, and the reaction was carried out as described above. For detection of CMV, GVA and TMV, reagents for reverse transcription were added directly to the tube with the CMV-specific primer, MulvRT (Fermentas); 1.5 μl from the reaction was taken for PCR reaction, using virus-specific primers. The PCR products were subjected to electrophoresis in 1% agarose gel in TAE buffer and stained with ethidium bromide (0.5 μg/ml) before photography. The same procedure was used with non-coated tubes. For immunodetection, the tube content was incubated with 10 μl SDS PAGE sample buffer (×4) for 10 min at 65° C.; collected and subjected to SDS PAGE.

### Screening for TYLCV Resistance

Transgenic and wild-type plants were kept in an insect-proof greenhouse at 19-25° C. Viruliferous whiteflies (following 24 h of acquisition access on TYLCV-infected tomatoes) were used to inoculate tomato plants (Zeidan M and Czosnek H, 1991, ibid). The insects were caged with plants (30-50 insects/plant) for a 48 h inoculation access feeding period. The whiteflies were discarded by spraying with imidacloprid. The plants were transferred to an insect-proof greenhouse to monitor appearance of symptoms. Severity of symptoms was monitored using the following disease severity index (DSI) (Lapidot M and Friedmann M, 2002, ibid): DSI=0: no visible symptoms; DSI=1: very slight yellowing of leaflet margin on apical leaf; DSI=2: some yellowing and minor curling of leaflet ends; DSI=3: leaf yellowing, curling and cupping, plant has reduced size but continues to develop; DSI=4: very severe stunting, yellowing, cupping and curling, plant cease to grow.

### Example 1

### Transformation and Regeneration of Plants

### Tomato Plants (“T₀” Generation)

Two different tomato lines, F1 and MP1, were chosen for this study to avoid genotype-related artifacts. These two genotypes are easy to transform and have high regeneration competence. A gene encoding a GroEL homologue from the whitefly B. tabaci endosymbiotic bacteria was cloned into an Agrobacterium binary vector, under the control of the promoter from the Arabidopsis phloem-specific sucrose transporter gene SUC2 (FIG. 1). The vector was introduced into Agrobacterium. Tomato cotyledons and hypocotyls were co-cultivated with the cells. Eighty nine independent tomato plants were regenerated from cotyledons and hypocotyls: 40 from F1 hypocotyls, 15 from F1 cotyledons, 27 from MP1 hypocotyls and 7 from MP1 cotyledons.

DNA extracted from each plant was subjected to PCR analysis using GroEL-specific primers as described hereinabove. Transgenic plants yielded the expected 500 base pair (bp) product. Of the 89 regenerated plants, a GroEL-specific PCR product was obtained from 24 plants only: 10 from F1 hypocotyls (plants number 1, 2, 4, 10, 14, 22, 34, 69, 71 and 86), 5 from F1 cotyledons (plants number 16, 26, 29, 35 and 43), 7 from MP1 hypocotyls (plants number 15, 28, 30, 45, 76, 80 and 82) and 2 from MP1 cotyledons (plants number 25 and 68). The nature of the PCR product was confirmed by hybridization with a GroEL-specific probe (FIG. 2A). The ratio of regenerated plants to transgenic plants was low because of the necessity to remove kanamycin selection during explant rooting, a fact that lead to a large number of escapes.

Tobacco (N. benthamiana) Plants

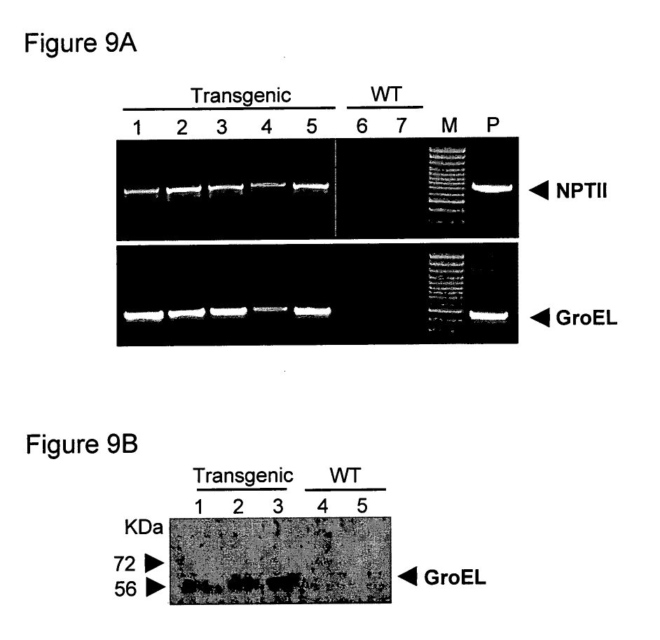

Tobacco plants were transformed with a whitefly GroEL gene, which was expressed under the control of the CaMV 35S promoter. The PCR and Western blot immuno-detection methods described hereinabove were used to demonstrate that the tobacco plants utilized in these studies were homozygous for the GroEL and NptII genes and expressed the GroEL protein (FIG. 9).

### Example 2

### Selection of Primary Transformants (“T₀” Generation) Expressing GroEL and Tolerant to Whitefly-Mediated TYLCV Inoculation

In order to select those transgenic plants that express GroEL, protein extracts from the leaves and the stems of plants of the “T₀” generation, were subjected to Western blot analysis. FIG. 2B shows the results for a selected number of plants, regenerated from cotyledons and hypocotyls of the F1 and MP1 genotypes. Transgenic plants 2, 25, 30 and 43 expressed the GroEL protein while the regenerated non-transgenic plant 44 did not. The GroEL protein was immunolocalized in tissue prints of stem sections and of young leaves, using an anti-GroEL antibody. Examination of the prints (FIG. 2C) indicated that GroEL was localized mainly in the inner phloem tissue of the GroEL PCR-positive transgenic tomato plants 30 and 43, but was not detected in non-transgenic tomato plant 44 regenerated from the same transformation events. The results obtained by the tissue prints and by the Western blots were totally compatible. All the twenty four regenerated plants that contained PCR-amplifiable GroEL DNA expressed the GroEL protein.

The twenty four “T₀” transgenic plants (from MP1 and F1) were challenged with TYLCV by caging plants with viruliferous whiteflies. Two weeks thereafter, DNA was extracted from a young leaflet sampled from each plant and was assayed for the presence of TYLCV DNA by PCR. The results shown in FIG. 3A indicate that at this early stage of infection the transgenic plants contained far less viral DNA compared to infected non-transgenic plants regenerated from tissue culture or from seeds. Severity of symptoms was scored 28, 40 and 60 days post inoculation (dpi): symptomless plants were given a disease severity index (DSI) of 0 (as non-inoculated plants), and fully symptomatic plants were scored as 4 (as inoculated non-transgenic plants). A graphic representation of the behavior of a randomly selected number of plants upon TYLCV inoculation is shown in FIG. 3B. Three of the 24 “T₀” transgenic plants (1, 26, 68) remained almost symptomless, even at 60 dpi, with a DSI of 0 to 1. Twenty one plants showed no or slight symptoms at 40 dpi (DSI of 1 to 2); the symptoms tended to improve or to disappear altogether thereafter (DSI of 0 to 1). Non-transgenic regenerated plants (e.g. 27 and 44) issued from the same transformation event presented typical TYLCV disease symptoms with a DSI of 4, as did susceptible control plants issued from seeds. At 90 dpi all the transgenic plants contained PCR-amplifiable viral DNA in similar amounts, excluding the possibility of infection escapes (not shown). An example of the appearance of the symptomless inoculated transgenic plants and of the symptomatic non-transgenic plants at 60 dpi is shown in FIG. 3C. In the greenhouse, infected symptomless transgenic tomato plants produced fruit yield comparable to the non infected non-transgenic plants of the same genotype. Infected non-transgenic plants did not produce fruit yield at all. Symptomless, or nearly-symptomless, infected tomato plants (DSI from 0 to 1) were considered as resistant, although they contained significant amounts of viral DNA.

### Example 3

### Production of the “T₁” Transgenic Generation: Integrity of the GroEL Gene Construct, Expression of the GroEL Protein and Resistance to TYLCV

The 24 primary transformed “T₀” plants were self-pollinated. Twenty seeds from nine randomly selected “T₀” plants were germinated in soil. All the 20 “T₁” progenies (named ‘A’ to ‘T’) from each of the nine primary “T₀” transformants were analyzed for the presence of the GroEL gene by PCR. Approximately 65 to 70% of the plants, regardless of the identity of the mother plant, contained amplifiable GroEL DNA, indicating that this generation segregated for the GroEL gene. Those plants that contained GroEL DNA were further investigated.

The integrity of the GroEL gene, flanked by the phloem-specific promoter and the NOS terminator, in the PCR-positive GroEL transgenic plants was confirmed by Southern blot hybridization of EcoRI-digested plant DNA (two EcoRI restriction sites encompass the SUC2-GroEL-NOS DNA, see FIG. 1). FIG. 4A shows that a unique DNA fragment of about 4,200 by hybridized with a probe consisting of the radiolabeled full-length GroEL gene. All plants that tested positive for GroEL DNA by PCR displayed the GroEL-containing 4,200 by DNA fragment. This fragment was absent in non-transgenic plants.

Presence of the GroEL protein was assayed by Western blot using the GroEL specific antibody. FIG. 4B shows the analysis of a number of selected plants. GroEL was detected in all of the transgenic plants assayed, but not in progeny of non-transgenic plants (e.g. plant 44D).

The transgenic plants from the “T₁” generation were tested for resistance to whitefly-mediated inoculation of TYLCV. As a first approach, 3 to 5 plants, progeny from each of the nine selected transgenic “T₀” parents which expressed the GroEL protein, were challenged with TYLCV as described hereinabove. Following whitefly-mediated inoculation, each plant was scored for symptoms and the DSI was noted at 30, 60 and 90 dpi. The progeny of the nine “T₀” transgenic parents showed very mild symptoms (DSI of 1 to 2) and either remained with this level of symptoms or the symptoms disappeared. The DSI within each group of plants was homogenous and varied only by one point, if at all (not shown). These results showed that all the transgenic plants which were resistant to TYLCV inoculation at the “T₀” generation (FIG. 3B) produced resistant “T₁” progeny plants.

The amounts of viral DNA in resistant transgenic plants of the “T₁” generation were compared with that of non-transgenic tomato plants, two months after inoculation (at 60 dpi). Semi-quantitative PCR was used, with β-actin as internal standard. FIG. 4C shows the results obtained with GroEL-expressing transgenic plants 15A and 30D, regenerated from MP1 cotyledons, which remained nearly symptomless upon inoculation. In plants 15A and 30D, as well as in the non-transgenic MP1 and FA144 varieties, the 410 by TYLCV amplicons was conspicuous after 14-16 cycles, while the 195 by actin amplicon was detected after 18-20 cycles. These results indicated that infected transgenic and non-transgenic plants contained about the same amount of viral DNA at 60 dpi, although the transgenic plants were virtually symptomless and the non-transgenic plants exhibited strong disease symptoms.

### Example 4

### Virus-GroEL Complexes in the Phloem of “T₁” Transgenic Tomato Plants

In the beginning of this study, it was postulated that GroEL expressed in the phloem of tomato plants will be able to trap virions, thereby preventing virus spread and infection. However similar amounts of viral DNA were present in symptomless transgenic plants and in symptomatic non-transgenic tomato (FIG. 4C). Such a result could be explained by the fact that in the resistant plants the virus was trapped by GroEL, all or part of it, and could not interact with plant factors to induce symptoms. Using a test previously described by Akad F et al. (2004, ibid), it was investigated whether GroEL-TYLCV complexes could be identified in infected transgenic tomato plants, or whether GroEL and TYLCV were not physically linked (FIG. 5). In these tests a TYLCV-specific PCR product was detectable only if a pre-existing TYLCV-GroEL complex would bind to anti-GroEL coated tubes (case A). No PCR product would be detected if a pre-existing TYLCV-GroEL complex was incubated with non-coated tubes (case B). Similarly no PCR product would be detected with sap from either non-infected transgenic plants (case C), infected non-transgenic plant (case D), and mixtures of sap from non-infected transgenic plants and sap from infected non-transgenic plant (case E).

PCR tubes were coated with anti-GroEL antibody; stem sap of infected (at 60 dpi) transgenic “T₁” plants 15A, 30B and 43C (with DSI close to 1) were added to the coated tube, as described hereinabove. After thorough washes, a PCR mix containing TYLCV-specific primers was added to the tube and the PCR products were analyzed. A virus-specific PCR product was detected indicating that pre-existing GroEL-TYLCV complexes did bind to the anti-GroEL-coated tubes (FIG. 5, case A). No PCR product was obtained when sap of plants 15A and 43C was added to uncoated tubes (case B). Similarly, no product was obtained when homogenates of non-inoculated transgenic plants 15D and 43G (case C), infected non-transgenic plants 11 and 12 (case D), or 1:1 sap mixes of non-infected transgenic plants 15D and 43G and infected non-transgenic plants 11 and 12 were incubated with the anti-GroEL coated tubes (case E).

These results indicated that GroEL expressed in the phloem of transgenic plants was able to produce a complex with virions, and that these complexes could be trapped by an anti-GroEL antibody. GroEL-TYLC complexes were not formed simply by mixing and incubating saps of non-infected transgenic plants and infected non-transgenic plants, possibly because of the low concentration of chaperon and virions in the sap of the respective plants and in the sap mixture. Thus, the results indicate that the GroEL-virion complexes need to be formed in vivo. The formation of the GroEL-TYLCV complex in the plant phloem might be the cause of the observed resistance observed in progeny of the transformed plants 2, 4, 15, 30 and 43.

### Example 5

### Production of “T₂” Transgenic Generation: Homozygosity for the GroEL Gene and Expression of the GroEL Protein

Two GroEL-expressing TYLCV-resistant transgenic plants of the “T₁” generation, 15A and 30D (FIG. 4) were self pollinated and 70 seeds from each plant were germinated to give “T₂” generation. Fifty six plants progeny of plant 15A and sixty five plants progeny of plant 30D were tested by PCR for the presence of GroEL DNA as well as for DNA of the NPTII selection marker. All the plants tested were positive for the two genes indicating that the 15A and 30D parents, and their progeny, were homozygous for GroEL and NPTII.

### Example 6

### Infected TYLCV-Resistant Plants as Inoculum Source for Whitefly-Mediated Transmission of Virus

It was further examined whether whitefly-mediated transmission of the virus from the infected transgenic plants was impacted by GroEL. To find out whether or not resistant transgenic plants can serve as a virus inoculum for whiteflies, insects were caged for a 72 h acquisition access period with: 1) two infected symptomless “T₂” tomatoes progeny of plant parent 15A (plants 3 and 7); 2) one infected symptomless “T₂” tomato progeny of parent 30D (plant 62); 3) one non-infected “T₂” tomato progeny of parent 30D (plant 68); and 4) one infected non-transgenic susceptible tomato (cv Daniella, FA144). PCR analyses indicated that the infected transgenic and non-transgenic plants contained viral DNA, while the non-infected transgenic control plant did not (FIG. 6A). The insects were collected separately from each plant source and each group was caged with ten non-transgenic non-infected susceptible test plants FA144. Following a 72 h inoculation access period, ten insects were collected from each plant group; PCR analyses indicated that these insects have acquired virus from the infected plants, whether transgenic or not (FIG. 6B). The plants were then treated with insecticide. Four weeks thereafter, all the test plants inoculated by insects fed on infected plants presented typical disease symptoms. PCR analyses indicated that the inoculated plants contained viral DNA (FIG. 6C). There was no difference in inoculation efficiency when either transgenic or non-transgenic tomato source plants served as virus source. Therefore, infected TYLCV-resistant transgenic plants may constitute a source of virus for whitefly-mediated transmission as effective as non-transgenic plants.

### Example 7

### Tolerance of GroEL-Expressing N. benthamiana Plants to Various Viruses

### GroEL Expressing Transgenic Plants are Tolerant to TYLCV and to CMV

It was previously shown that TYLCV and CMV bind to GroEL in vitro, while GVA and TMV do not (Akad F et al., 2004, ibid). Thus, it was further examined whether a transgenic plants expressing GroEL would be tolerant to any GroEL-binding virus. Transgenic and non-transgenic N. benthamiana plants produced as described hereinabove were inoculated with TYLCV and with CMV. Plants were inoculated with TYLCV using viruliferous whiteflies, and mechanically with CMV. TYLCV infection was assessed by PCR and by Western blot analysis 7 days after inoculation (dpi) (FIG. 10A), while CMV infection was assessed after 5 dpi (FIG. 11A).

First, the presence of GroEL-TYLCV and GroEL-CMV complexes was examined in the infected transgenic N. benthamiana. In these tests, sap of infected transgenic plants was incubated in PCR tubes coated with anti-GroEL antibody as described hereinabove. Following incubation of sap of infected transgenic N. benthamiana plants in anti-GroEL coated tubes, TYLCV and CMV were detected by PCR (and RT-PCR) and by anti-CP antibodies, demonstrating that these viruses were bound to GroEL in the plant sap. These two viruses were not detected when sap of infected non-transgenic tobacco plants were tested in the same experiments (FIGS. 10B and 11B).

Non-transgenic N. benthamiana inoculated with TYLCV showed disease symptoms starting at 7-10 dpi, which included leaf curling and cessation of growth. The symptoms worsened with time. In comparison, TYLCV-inoculated transgenic plants remained symptomless for at least 30 days after inoculation, and then presented either mild or no symptoms (FIG. 10C). Typical CMV disease symptoms started to appear at 5-7 dpi in CMV inoculated non-transgenic plants and worsened rapidly thereafter. The plants remained stunted and mottled. By comparison, transgenic tobacco remained almost symptomless for at least three months after inoculation and flowered similarly to non-inoculated wild type plants (shown at 100 dpi, FIG. 11C).

### GroEL-Expressing Plants are Susceptible to GVA and TMV

Transgenic and non-transgenic N. benthamiana plants were mechanically inoculated with GVA and with TMV. Infection was assessed by RT-PCR and by Western blot analyses at 5-7 dpi (FIGS. 12A and 13A). The possible binding of GVA and TMV particles to GroEL in transgenic N. benthamiana plants was assessed as described above. Sap of infected transgenic plants at 7 dpi was incubated in PCR tubes coated with anti-GroEL antibody. Binding of GroEL-virus complexes to the coated tubes was assayed by RT-PCR and with virus-specific antibodies. Both viruses were undetectable, indicating that, contrary to TYLCV and CMV, GVA and TMV were not bound to GroEL in the sap of infected transgenic plants (FIGS. 12B and 13B). Following inoculation with GVA and with TMV, the transgenic N. benthamiana plants presented symptoms identical to those conspicuous in infected not transgenic tobacco plants (FIGS. 12C and 13C). Typical disease symptoms started to appear at 14 dpi in GVA inoculated plants and worsened thereafter. TMV inoculated plants died within the first week after inoculation.

### Example 8

### Non-Transgenic Susceptible Tomato Plants Grafted on Resistant GroEL-Transgenic Scions

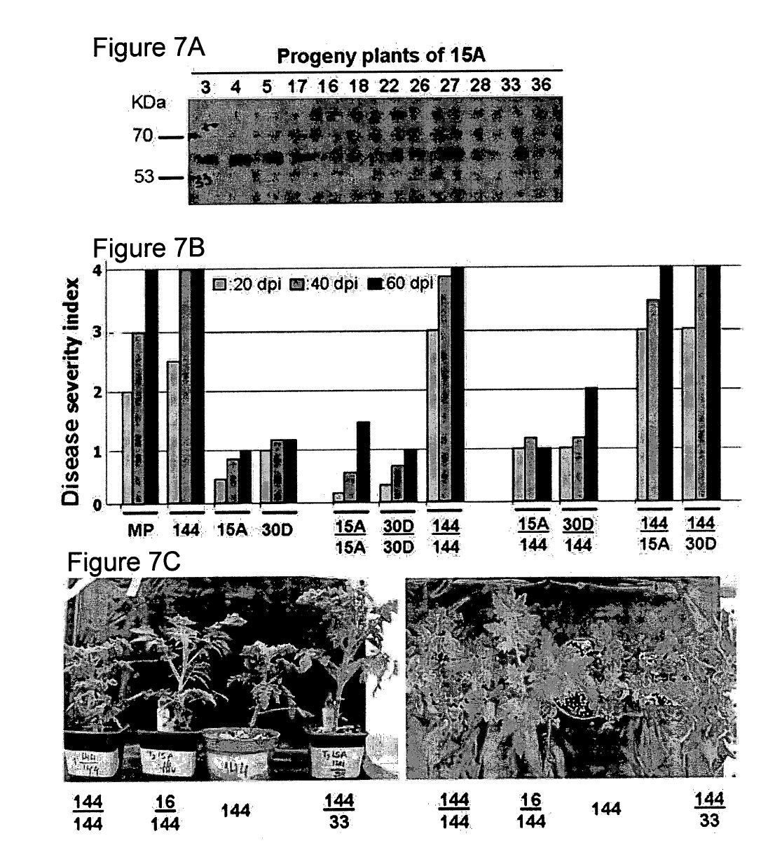

It was investigated whether GroEL can move long-distance from TYLCV-resistant transgenic scion to TYLCV-susceptible non-transgenic graft. Progeny plants of parent 15A and 30D (6 weeks after sowing) were used as scions on which susceptible FA144 plants were grafted. One week before grafting the presence of GroEL was confirmed in the scions plant source (FIG. 7A). GroEL-expressing progeny of plants 15A and 30D were also grafted on FA144 susceptible scions. As controls, FA144 plants and progeny of 15A and 30D plants grafted on themselves, and non-grafted resistant and susceptible plants were used. Two weeks after grafting, the plants were inoculated with viruliferous whiteflies as described hereinabove. DSI was recorded 20, 40, 60 and 80 days after inoculation. The results summarized in FIG. 7B show that the FA144 plants grafted on resistant 15A and 30D progeny plants showed viral disease symptoms comparable to those of FA144 plants grafted on themselves or on non-grafted FA144 plants In comparison, 15A and 30D progeny plants grafted on FA144 plants retained their resistance to the virus and in this respect behaved similarly to 15A and 30D grafted on themselves or to non-grafted 15A and 30D progeny plants. PCR analyses performed one week after inoculation indicated that all the plants contained TYLCV DNA (scions and grafts, or whole plants) and that there was no escape from inoculation.

In the light of these results, it was further investigated whether the symptomatic FA144 grafts contained GroEL-TYLCV complexes 60 days after inoculation.

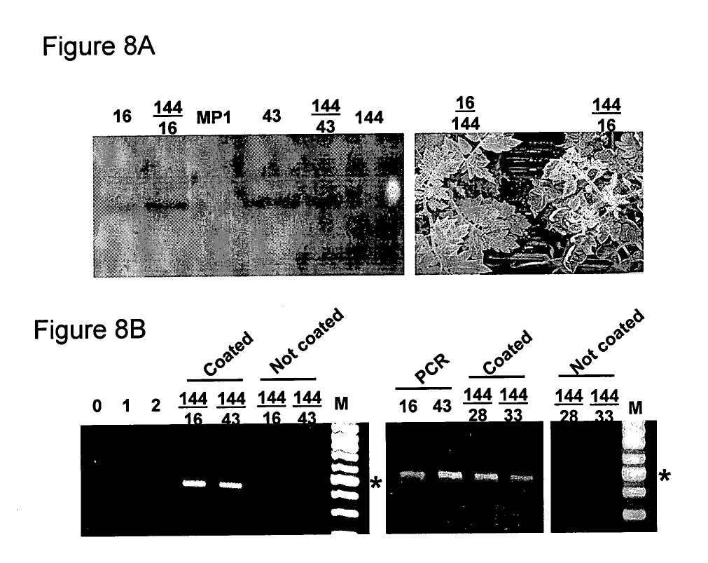

Western blot analysis showed that GroEL did translocate from scions of GroEL-expressing plants into susceptible grafts. FIG. 8A shows that GroEL present in the nearly symptomless (DSI of about 1) plants 16 (progeny of 15A) and 43 (progeny of 30D) was also conspicuous in the symptomatic FA144 plants grafted on the 16 and 43 scions. The presence of virions bound to GroEL was investigated as described above. Plant sap was incubated in PCR tubes coated with anti-GroEL antibody. After washing, a PCR mix containing TYLCV-specific primers was added and the PCR products were analyzed. FIG. 8B shows that a virus-specific PCR product was detected in the FA144 plants grafted on transgenic plants 16, 28 and 33 (progeny of 15A) and 43 (progeny of 30D), indicating that GroEL-TYLCV complexes were present in the sap of the infected symptomatic FA144 grafts. All the FA144 plants grafted on GroEL-transgenic scions gave identical results. No PCR product was obtained when the sap of the FA144 scions was incubated in tubes not coated with GroEL antibody. Hence, despite the fact that FA144 grafts were still susceptible to viral infection (FIG. 7C), the presence of GroEL translocated from the transgenic scion into the non-transgenic grafts and its ability to bind virions, indicates that it may be possible to obtain tolerant engrafted plants. This may require using as scions more established plants comprising at least several leaves.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A transgenic plant comprising at least one cell transformed with a polynucleotide encoding a GroEL protein, an active fragment, variant or homologue thereof.

2. The transgenic plant of claim 1, wherein the GroEL protein, the active fragment, variant or homologue thereof is capable of binding a plant virus.

3. The transgenic plant of claim 2, wherein the plant virus has a globular shape and a capsid protein (CP) characterized by a basic isoelectric point, a marked positive charge and a high percentage of arginine residues.

4. The transgenic plant of claim 1, wherein the polynucleotide encodes a GroEL protein of an endosymbiotic bacterium of Bemisia tabaci.

5. The transgenic plant of claim 1, wherein the GroEL protein has an amino acid sequence as set forth is SEQ ID NO:2.

6. The transgenic plant of claim 1, wherein the polynucleotide comprises a nucleic acid sequence having at least 70% homology to SEQ ID NO:1.

7. The transgenic plant of claim 6, wherein the polynucleotide comprises a nucleic acid sequence as set forth in SEQ ID NO:1.

8. The transgenic plant of claim 1, wherein the polynucleotide encodes a GroEL fragment having a length of at least 10 amino acids.

9. The transgenic plant of claim 1, wherein the GroEL protein or the active fragment, variant or homologue thereof is expressed in said plant in a tissue-specific manner.

10. The transgenic plant of claim 9, wherein the GroEL protein or the active fragment, variant or homologue thereof is expressed in the phloem.

11. The transgenic plant of claim 1, wherein said plant has an enhanced tolerance to at least one disease caused by an insect-transmitted plant pathogenic virus compared to a non-transgenic plant, wherein the insect-transmitted plant pathogenic virus is selected from the group presented in Table 1.

12. (canceled)

13. The transgenic plant of claim 11, wherein the insect-transmitted plant pathogenic virus is selected from the group consisting of TYLCV and CMV.

14. The transgenic plant of claim 1, wherein said plant is selected from the group consisting of tomato, tobacco, cucumber, prune, potato, bean, barley, soybean, pea, beet, grapevine, petunia, abutilon, melon, watermelon, okra, cotton, cassava, wheat, maize, rice and cabbage.

15. A plant seed produced by the transgenic plant of claim 1, wherein a plant grown from said seed has an enhanced tolerance to at least one disease caused by an insect-transmitted plant pathogenic virus compared to a non-transgenic plant.

16. (canceled)

17. A tissue culture comprising at least one transformed cell of claim 1 or a protoplast derived therefrom, wherein the tissue culture regenerates plants having an enhanced tolerance to at least one disease caused by an insect-transmitted plant pathogenic virus compared to a non-transgenic plant.

18. (canceled)

19. A plant regenerated from the tissue culture of claim 17.

20. An engrafted plant comprising as a rootstock a transgenic plant according to claim 1 or a part thereof and a non-transgenic graft, wherein the entire engrafted plant has an enhanced tolerance to at least one disease caused by an insect-transmitted plant pathogenic virus.

21. The engrafted plant of claim 20, wherein the insect-transmitted plant pathogenic virus is selected from the group presented in Table 1.

22. The engrafted plant of claim 21, wherein the insect-transmitted plant pathogenic virus is selected from the group consisting of TYLCV and CMV.

23. The engrafted plant of claim 20, wherein the rootstock and the graft are of the same plant variety or of different plant varieties.

24. (canceled)

25. A method of producing a transgenic plant having an enhanced tolerance to at least one disease caused by an insect-transmitted plant pathogenic virus comprising (a) transforming a plant cell with a polynucleotide encoding GroEL protein or an active fragment, variant or homologue thereof; and (b) regenerating the transformed cell into a plant, wherein the regenerated plant has an increased tolerance to at least one disease caused by the insect-transmitted plant pathogenic virus as compared to a corresponding non-transgenic plant.

26. (canceled)

27. (canceled)

28. The method of claim 25, wherein the polynucleotide encodes a GroEL protein of an endosymbiotic bacterium of Bemisia tabaci.

29. The method of claim 25, wherein the GroEL protein has an amino acid sequence as set forth is SEQ ID NO:2.

30. The method of claim 25, wherein the polynucleotide comprises a nucleic acid sequence having at least 70% homology to SEQ ID NO:1.

31. (canceled)

32. (canceled)

33. The method of claim 25, wherein the polynucleotide further comprises a regulatory element selected from the group consisting of an enhancer, a promoter, and a transcription termination sequence.

34. The method of claim 33, wherein the promoter is selected from the group consisting of a constitutive prompter, an induced promoter and a tissue-specific promoter.

35. The method of claim 34, wherein the promoter is a phloem specific promoter.

36. (canceled)

37. (canceled)

38. The method of claim 25, wherein the regenerated plant expresses the GroEL protein, fragment, variant or homologue thereof.

39. The method of claim 25, wherein the regenerated plant has an increased tolerance to an insect-transmitted plant pathogenic virus selected from the group presented in Table 1.

40. (canceled)

41. A plant produced by the method of claim 25.

42. A method of conferring to a plant an enhanced tolerance to an insect-transmitted plant pathogenic virus, comprising (a) providing a transgenic rootstock comprising at least one cell transformed with a polynucleotide encoding GroEL protein, or an active fragment, variant or homologue thereof and (b) grafting the plant or part thereof onto the transgenic rootstock as to obtain an engrafted plant having an enhanced tolerance to at least one disease caused by an insect-transmitted plant pathogenic virus.

43. (canceled)

44. (canceled)

45. The method of claim 42, wherein the polynucleotide encodes a GroEL protein of an endosymbiotic bacterium of Bemisia tabaci.

46. The method of claim 45, wherein the GroEL protein has an amino acid sequence as set forth is SEQ ID NO:2.

47. The method of claim 42, wherein the polynucleotide comprises a nucleic acid sequence having at least 70% homology to SEQ ID NO:1.

48. (canceled)

49. (canceled)

50. The method of claim 42, wherein the transgenic rootstock expresses the GroEL protein, fragment, variant or homologue thereof.

51. The method of claim 42, wherein the engrafted plant has an increased tolerance to an insect-transmitted plant pathogenic virus selected from the group presented in Table 1.

52. (canceled)

53. The method of claim 42, wherein the transgenic rootstock and the plant are of the same variety or of different varieties.

54. (canceled)


SEQUENCE LISTING (22 sequences)

SEQ ID NO: 1 - DNA, 1668 bp, Bemisia tabaci
atggcagctaaagacttaaaatttggtaatgacgcccgaaaaaaaatgttaaagggtgtcaacattctggctaatgcagtaaaggtgactttgggtcccaaaggtcgcaatgttgtattagataaatcttatggagcgccttctattactaaagacggtgtttctgtcgcccgtgaaattgagctggaagacaaatttgaaaacatgggtgctcaaatggtcaaagaagtcgcctctaaagcaaacgatgcggcaggagacggcaccaccacagcgaccgtgttagcacaatccatcgtcaccgaggggttaaaggcagttgcagcaggcatgaatccaatggatcttaagcgtggtatcgataaagcagtagaagccgctgttgaagaactcaaaattatatctaaaccttgcaaagactctaaagaaattgcacaggtaggcactatctctgccaatgcagatgcaaaagtaggtaaattaatttcagacgcaatgaagagggttactaacgaaggcgtgatcactgttgaagaagcctctggcctggaagacgacctcatcgttgtggaaggaatgcagtttgatcgcggttatctttctccttattttattaacaagcaagaaagcagctctattgaatttgacaacccttacatcttattagttgataaaaaaatctctaacatcagagacatgttatccatattagaagtggtggcaaaagaagggaagcctttattaattatcgcagaagatgttgaaggtgaagctttggcaaccttggtggtcaatacgatgagaggcatcgtcaaggtgtctgctgtcaaggccccgggttttggagatcgccgcaaagagatgctacaggatattgcagtattaacgcacggtcatgtgatttcagaagaaacaggagattctttagaaaaggcaaccaaagaaaatctgggcaatgcaaaacgcgtcgtcattactaaagatgcaaccactattattgatggagcaggtgaaaaatcgaagatcgaggctcgtattcaaaatatcaagaagcagattgaaaatgcgacttctgattatgataaagaaaaactgcaagagcgtgttgctaaattgtcagggggagtcgctgttataaaagtgggcgctccgaccgaaattgcgatgaaagagaaaaaagcccgtgtggaagatgccttacaggcaacaagagcagcagtagaagaaggggttgttccaggcggcggtgttgctttgatacgtgtcgcctccaaaattgcaaattctagtttaaaaggggataacgaagatcaaaatgtgggtattcgtgttgctcttcgtgccatggagtctcctctgcgtcagattgtggtcaatgctggagaagaagcttctgtgattgcaaataaaattaaagaaaacaaaggaaatgacaactacggctataatgcgcaaacagaagcttatggtgacatgattgagatgggaattttagatcctactaaagtgacacgttctgcattgcaatatgccgcttctatcgcaggattaatgataacgactgaatgtatggtcactgatttaccgaaagaagaaaaagcctctgatatgggtgctggtggaatgggtggaataggtggaatgggcggaatgaatggtatgatgtaa

SEQ ID NO: 2 - protein, 555 aa, Bemisia tabaci
MAAKDLKFGNDARKKMLKGVNILANAVKVTLGPKGRNVVLDKSYGAPSITKDGVSVAREIELEDKFENMGAQMVKEVASKANDAAGDGTTTATVLAQSIVTEGLKAVAAGMNPMDLKRGIDKAVEAAVEELKIISKPCKDSKEIAQVGTISANADAKVGKLISDAMKRVTNEGVITVEEASGLEDDLIVVEGMQFDRGYLSPYFINKQESSSIEFDNPYILLVDKKISNIRDMLSILEVVAKEGKPLLIIAEDVEGEALATLVVNTMRGIVKVSAVKAPGFGDRRKEMLQDIAVLTHGHVISEETGDSLEKATKENLGNAKRVVITKDATTIIDGAGEKSKIEARIQNIKKQIENATSDYDKEKLQERVAKLSGGVAVIKVGAPTEIAMKEKKARVEDALQATRAAVEEGVVPGGGVALIRVASKIANSSLKGDNEDQNVGIRVALRAMESPLRQIVVNAGEEASVIANKIKENKGNDNYGYNAQTEAYGDMIEMGILDPTKVTRSALQYAASIAGLMITTECMVTDLPKEEKASDMGAGGMGGIGGMGGMNGMM

SEQ ID NO: 3 - DNA, 30 bp, Artificial Sequence
  Synthetic primer
tttcatgacagctaaagacttaaaatttgg

SEQ ID NO: 4 - DNA, 31 bp, Artificial Sequence
  Synthetic primer
tgagctcttacatcataccattcattccgcc

SEQ ID NO: 5 - DNA, 21 bp, Artificial Sequence
  Synthetic primer
tctagaatggcagctaaagac

SEQ ID NO: 6 - DNA, 22 bp, Artificial Sequence
  Synthetic primer
gagctcttacatcataccattc

SEQ ID NO: 7 - DNA, 23 bp, Artificial Sequence
  Synthetic primer
gcagaagatgttgaaggtgaagc

SEQ ID NO: 8 - DNA, 24 bp, Artificial Sequence
  Synthetic primer
ccttcttctactgctgcttcttgt

SEQ ID NO: 9 - DNA, 22 bp, Artificial Sequence
  Synthetic primer
gccgcttgggtggagaggctat

SEQ ID NO: 10 - DNA, 22 bp, Artificial Sequence
  Synthetic primer
gaggaagcggtcagcccattcg

SEQ ID NO: 11 - DNA, 20 bp, Artificial Sequence
  Synthetic primer
atacttggacacctaatggc

SEQ ID NO: 12 - DNA, 17 bp, Artificial Sequence
  Synthetic primer
agtcacgggcccttaca

SEQ ID NO: 13 - DNA, 20 bp, Artificial Sequence
  Synthetic primer
ggaaaagcttgcctatgtgg

SEQ ID NO: 14 - DNA, 20 bp, Artificial Sequence
  Synthetic primer
cctgcagcttccataccaat

SEQ ID NO: 15 - DNA, 19 bp, Artificial Sequence
  Synthetic primer
gaaggctgaacttcgacag

SEQ ID NO: 16 - DNA, 21 bp, Artificial Sequence
  Synthetic primer
attgggctgtttccatagggc

SEQ ID NO: 17 - DNA, 22 bp, Artificial Sequence
  Synthetic primer
taactcaacagtcctcagcggc

SEQ ID NO: 18 - DNA, 22 bp, Artificial Sequence
  Synthetic primer
ctgacggttttgtttgctcagc

SEQ ID NO: 19 - DNA, 18 bp, Artificial Sequence
  Synthetic primer
gacaaatggcacactacg

SEQ ID NO: 20 - DNA, 21 bp, Artificial Sequence
  Synthetic primer
aagcctgacctagtcatcttg

SEQ ID NO: 21 - DNA, 27 bp, Artificial Sequence
  Synthetic primer
atgtcttacagtatcactactccatat

SEQ ID NO: 22 - DNA, 23 bp, Artificial Sequence
  Synthetic primer
caagttgcaggaccagaggtcca